(19) United States
(12) Patent Application Publication
(10) Pub. No.: US 2009/0227674 A1
(43) Pub. Date: Sep. 10, 2009

(54) Combination methods fo saha and targretin for treating cancer

(76) Inventors: Victoria M. Richon, Wellesley, MA (US); Stanley R. Frankel, Yardley, PA (US); Steven Averbuch, North Wales, PA (US)

Correspondence Address: MERCK AND CO., INC, P O BOX 2000, RAHWAY, NJ, 07065-0907, US

(21) Appl. No.: 11/990,724

(22) PCT Filed: Aug. 18, 2006
(86) PCT No.: PCT/US06/32282

§ 371 Date: Mar. 20, 2009

Related U.S. Application Data
(60) Provisional application No. 60/709,599, filed on Aug. 18, 2005.
(60) Provisional application No. 60/733,752, filed on Nov. 4, 2005.

(51) Int. Cl. A61K 31/216; A61K 31/192; A61P 35/00

(52) U.S. Cl. 514/545; 514/569

(57) ABSTRACT

The present invention relates to a method of treating cancer in a subject in need thereof, by administering to a subject in need thereof a first amount of SAHA or a pharmaceutically acceptable salt or hydrate thereof, and a second amount of Targretin. The SAHA and Targretin may be administered to comprise therapeutically effective amounts.

al. (1996) PNAS 93:5705-8). These compounds are targeted towards mechanisms inherent to the ability of a neoplastic cell to become malignant, as they do not appear to have toxicity in doses effective for inhibition of tumor growth in animals (Cohen, L. A., Amin, S., Marks, P. A., Rifkind, R. A., Desai, D., and Richon, V. M. (1999) Anticancer Research 19:4999-5006). There are several lines of evidence that histone acetylation and deacetylation are mechanisms by which transcriptional regulation in a cell is achieved (Grunstein, M. (1997) Nature 389:349-52). These effects are thought to occur through changes in the structure of chromatin by altering the affinity of histone proteins for coiled DNA in the nucleosome.

There are five types of histones that have been identified (designated H1, H2A, H2B, H3 and H4). Histones H2A, H2B, H3, and H4 are found in the nucleosomes and H1 is a linker located between nucleosomes. Each nucleosome contains two of each histone type within its core, except for H1, which is present singly in the outer portion of the nucleosome structure. It is believed that when the histone proteins are hypoacetylated, there is a greater affinity of the histone to the DNA phosphate backbone. This affinity causes DNA to be tightly bound to the histone and renders the DNA inaccessible to transcriptional regulatory elements and machinery. The regulation of acetylated states occurs through the balance of activity between two enzyme complexes, histone acetyl transferase (HAT) and histone deacetylase (HDAC). The hypoacetylated state is thought to inhibit transcription of associated DNA. This hypoacetylated state is catalyzed by large multiprotein complexes that include HDAC enzymes. In particular, HDACs have been shown to catalyze the removal of acetyl groups from the chromatin core histones.

Retinoids affect gene expression by binding to nuclear retinoid receptors and their coregulators, leading to transcriptional activation of target genes that ultimately control growth and differentiation (see, e.g., Ralhan and Kaur, 2003, J. Biol. Regul. Homost. Agents 17(1):66-91). There are two functionally distinct classes of nuclear retinoid receptors: retinoic acid receptors (RAR) and retinoid X receptors (RXR). Each of the retinoid receptor classes includes three subtypes designated α, β, and γ, which are encoded by distinct genes (Chambers, 1996, FASEB J. 10:940-54). The RARs bind both all-trans retinoic acid (ATRA) and 9-cis-retinoic acid (9-cis-RA), whereas the RXRs bind only 9-cis-RA. These receptors also bind to a variety of synthetic retinoids. RARs can form heterodimers with RXRs, and RXRs can also form homodimers that bind to specific segments of DNA, called retinoic acid response elements (RARE) and retinoid X response elements (RXRE), respectively (see, e.g., Ralhan and Kaur, 2003, J. Biol. Regul Homost. Agents 17(1):66-91). 3-methyl TTNEB (e.g., Bexarotene; Targretin®) is a highly selective synthetic RXR agonist. It is generally believed that retinoids cause apoptosis and regulate cell growth through receptor-mediated effects on gene expression.

Besides the aim to increase the therapeutic efficacy, another purpose of combination treatment is the potential decrease of the doses of the individual components in the resulting combinations in order to decrease unwanted or harmful side effects caused by higher doses of the individual components. Thus, there is an urgent need to discover suitable methods for the treatment of cancer, including combination treatments that result in decreased side effects and that are effective at treating and controlling malignancies.

### SUMMARY OF THE INVENTION

The present invention is based on the discovery that histone deacetylase (HDAC) inhibitors, for example suberoylanilide hydroxamic acid (SAHA), can be used in combination with a retinoid agent, for example Targretin, and optionally another anti-cancer agent, to provide therapeutic efficacy.

The invention relates to a method for treating cancer or other disease comprising administering to a subject in need thereof an amount of an HDAC inhibitor, e.g., SAHA, and an amount of a retinoid agent, for example Targretin, and optionally another anti-cancer agent.

The invention further relates to pharmaceutical combinations useful for the treatment of cancer or other disease comprising an amount of an HDAC inhibitor, e.g., SAHA, and an amount of a retinoid agent, for example Targretin.

In one embodiment, the pharmaceutical compositions of the present invention can comprise a histone deacetylase inhibitor, e.g., SAHA, represented by the structure:

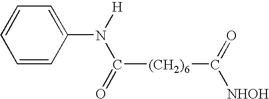

or a pharmaceutically acceptable salt or hydrate thereof, and a retinoid agent, 4-[1-(5,6,7,8-Tetrahydro-3,5,5,8,8-pentamethyl-2-naphthalenyl)ethenyl]benzoic acid (3-methyl TTNEB) (Targretin), represented by the structure:

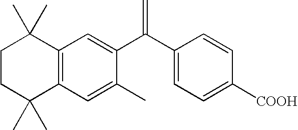

or a pharmaceutically acceptable salt or hydrate thereof.

The compositions of the present invention can be formulated for oral administration and can comprise, inter alia, 100 mg of SAHA and 75 mg of Targretin.

The invention further relates to the use of an amount of an HDAC inhibitor, e.g., SAHA, and an amount of a retinoid agent, for example Targretin, and optionally another anti-cancer agent, for the manufacture of one or more medicaments for treating cancer or other disease.

In further embodiments, the HDAC inhibitors suitable for use in the present invention include but are not limited to hydroxamic acid derivatives, Short Chain Fatty Acids (SCFAs), cyclic tetrapeptides, benzamide derivatives, or electrophilic ketone derivatives.

In further embodiments, the treatment procedures are performed sequentially in any order, alternating in any order, simultaneously, or any combination thereof. In particular, the administration of an HDAC inhibitor, the administration of the retinoid agent, and optionally another anti-cancer agent can be performed concurrently, consecutively, or e.g., alternating concurrent and consecutive administration. For example, in one embodiment, the HDAC inhibitor, e.g., SAHA, is administered prior to administering the retinoid agent, e.g., Targretin. In other embodiments, the HDAC inhibitor and the retinoid agent are administered orally.

In another embodiment, the HDAC inhibitor, e.g., SAHA, can be pre-administered 1 week prior to a concurrent administration of HDAC inhibitor and retinoid agent, e.g., Targretin, where SAHA is pre-administered or concurrently administered at 400 mg per day. The concurrent administration of SAHA and Targretin can be for six 28-day cycles, or alternatively, SAHA can be administered 400 mg once a day for six 28-day cycles, Targretin can be administered at 150 mg per day for the first 28-day cycle, and at 225 mg per day for the second to sixth 28-day cycle.

In other embodiments, SAHA and Targretin can be concurrently administered, wherein SAHA is administered 400 mg once a day for six 28-day cycles, Targretin is administered at 150 mg per day for the first 28-day cycle, at 225 mg per day for the second 28-day cycle, and at 300 mg per day for the third to sixth 28-day cycle.

SAHA and Targretin, in further embodiments, can be concurrently administered wherein SAHA is administered 400 mg once a day for six 28-day cycles, Targretin is administered at 150 mg per day for the first 28-day cycle, at 300 mg per day for the second 28-day cycle, and at 375 mg per day for the third to sixth 28-day cycle.

In other embodiments, SAHA and Targretin can be concurrently administered wherein SAHA is administered 400 mg once a day for six 28-day cycles, Targretin is administered at 150 mg per day for the first 28-day cycle, at 300 mg per day for the second 28-day cycle, and at 450 mg per day for the third to sixth 28-day cycle.

In further embodiments, a lipid-lowering agent can be administered during or before the pre-administration period, or a combination thereof. The lipid-lowering agent can be, for example, fenofibrate. Alternatively, thyroxine can be administered at the start of the concurrent administration period. The thyroxine can be, but is not limited to, levothyroxine.

In further embodiments, the additional anti-cancer agent can be an alkylating agent, an antibiotic agent, an antimetabolic agent, a hormonal agent, a plant-derived agent, an anti-angiogenic agent, a differentiation inducing agent, a cell growth arrest inducing agent, an apoptosis inducing agent, a cytotoxic agent, a biologic agent, a gene therapy agent, a retinoid agent, or any combination thereof.

In further embodiments, the combination therapy of the invention is used to treat inflammatory diseases, autoimmune diseases, allergic diseases, diseases associated with oxidative stress, neurodegenerative diseases, and diseases characterized by cellular hyperproliferation (e.g., cancers), or any combination thereof.

In further embodiments, the combination therapy is used to treat diseases such as cancer including, without limitation, leukemia, lymphoma, myeloma, sarcoma, carcinoma, solid tumor, or any combination thereof. The cancer can be, for example, a cutaneous T-cell lymphoma (CTCL).

These and other embodiments are encompassed by the following Detailed Description.

### BRIEF DESCRIPTION OF THE DRAWINGS

The foregoing and other objects, features and advantages of the invention will be apparent from the following more particular description of various embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A-1B: Effect of Vorinostat and Targretin® combination in an HH cell line. FIG. 1A: Cells were left untreated, treated with 0.37 μM Vorinostat, treated with 0.60 μM Targretin®, or treated with a combination of 0.37 μM Vorinostat and 0.60 μM Targretin® as described in Example 2. FIG. 1B: Cells were left untreated, treated with 1 μM Vorinostat, treated with 10 μM Targretin®, or treated with a combination of 1 μM Vorinostat and 10 μM Targretin® as described in Example 2.

### DETAILED DESCRIPTION OF THE INVENTION

The present invention relates to a method of treating cancer or other disease, in a subject in need thereof, by administering to a subject in need thereof an amount of an HDAC inhibitor or a pharmaceutically acceptable salt or hydrate thereof, in a treatment procedure, and an amount of one or more anti-cancer agents (e.g., retinoid agents) in another treatment procedure, wherein the amounts together comprise a therapeutically effective amount. The invention further relates to a method of treating cancer or other disease, in a subject in need thereof, by administering to a subject in need thereof an amount of suberoylanilide hydroxamic acid (SAHA) or a pharmaceutically acceptable salt or hydrate thereof, in a treatment procedure, and an amount of one or more anti-cancer agents (e.g., retinoid agents) in another treatment procedure, wherein the amounts can comprise a therapeutically effective amount. The effect of SAHA in combination with a retinoid agent such as Targretin, and optionally another anti-cancer agent can be, e.g., additive or synergistic.

In one aspect, the method comprises administering to a patient in need thereof a first amount of a histone deacetylase inhibitor, e.g., SAHA or a pharmaceutically acceptable salt or hydrate thereof, in a first treatment procedure, and a second amount of an anti-cancer agent, such as a retinoid agent, e.g., 3-methyl TTNEB (“Targretin”; bexarotene), or a pharmaceutically acceptable salt or hydrate thereof, in a second treatment procedure, and optionally a third amount of another anti-cancer agent, or a pharmaceutically acceptable salt or hydrate thereof, in a third treatment procedure. The first and second, and optionally third treatments can comprise a therapeutically effective amount.

The invention further relates to pharmaceutical combinations useful for the treatment of cancer or other disease. In one aspect, the pharmaceutical combination comprises a first amount of an HDAC inhibitor, e.g., SAHA or a pharmaceutically acceptable salt or hydrate thereof, and a second amount of an anti-cancer agent, such as a retinoid agent, e.g., 3-methyl TTNEB, or a pharmaceutically acceptable salt or hydrate thereof, and optionally, a third amount of another anti-cancer agent, or a pharmaceutically acceptable salt or hydrate thereof. The first and second and optional third amounts can comprise a therapeutically effective amount.

The invention further relates to the use of an amount of an HDAC inhibitor and an amount of an anti-cancer agent, such as a retinoid agent, e.g., 3-methyl TTNEB, and optionally another anti-cancer agent, for the manufacture of a medicament for treatment of cancer or other disease. In one aspect, the medicament comprises a first amount of an HDAC inhibitor, e.g., SAHA or a pharmaceutically acceptable salt or hydrate thereof, and a second amount of an anti-cancer agent, e.g., 3-methyl TTNEB, or a pharmaceutically acceptable salt or hydrate thereof, and optionally a third amount of another anti-cancer agent, or a pharmaceutically acceptable salt or hydrate thereof.

### DEFINITIONS

The term “treating” in its various grammatical forms in relation to the present invention refers to preventing (i.e. chemoprevention), curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease. Because some of the inventive methods involve the physical removal of the etiological agent, the artisan will recognize that they are equally effective in situations where the inventive compound is administered prior to, or simultaneous with, exposure to the etiological agent (prophylactic treatment) and situations where the inventive compounds are administered after (even well after) exposure to the etiological agent.

Treatment of cancer, as used herein, refers to partially or totally inhibiting, delaying or preventing the progression of cancer including cancer metastasis; inhibiting, delaying or preventing the recurrence of cancer including cancer metastasis; or preventing the onset or development of cancer (chemoprevention) in a mammal, for example a human. In addition, the method of the present invention is intended for the treatment of chemoprevention of human patients with cancer. However, it is also likely that the method would be effective in the treatment of cancer in other mammals.

The “anti-cancer agents” of the invention encompass those described herein, including any pharmaceutically acceptable salts or hydrates of such agents, or any free acids, free bases, or other free forms of such agents, and as non-limiting examples: A) Polar compounds (Marks et al. (1987); Friend, C., Scher, W., Holland, J. W., and Sato, T. (1971) Proc. Natl. Acad. Sci (USA) 68: 378-382; Tanaka, M., Levy, J., Terada, M., Breslow, R., Rifkind, R. A., and Marks, P. A. (1975) Proc. Natl. Acad. Sci (USA) 72: 1003-1006; Reuben, R. C., Wife, R. L., Breslow, R., Rifkind, R. A., and Marks, P. A. (1976) Proc. Natl. Acad. Sci (USA) 73: 862-866); B) Derivatives of vitamin D and retinoic acid (Abe, E., Miyaura, C., Sakagami, H., Takeda, M., Konno, K., Yamazaki, T., Yoshika, S., and Suda, T. (1981) Proc. Natl. Acad. Sci (USA) 78: 4990-4994; Schwartz, E. L., Snoddy, J. R., Kreutter, D., Rasmussen, H., and Sartorelli, A. C. (1983) Proc. Am. Assoc. Cancer Res. 24:18; Tanenaga, K., Hozumi, M., and Sakagami, Y. (1980) Cancer Res. 40: 914-919); C) Steroid hormones (Lotem, J. and Sachs, L. (1975) Int. J. Cancer 15: 731-740); D) Growth factors (Sachs, L. (1978) Nature (Lond.) 274: 535, Metcalf, D. (1985) Science, 229: 16-22); E) Proteases (Scher, W., Scher, B. M., and Waxman, S. (1983) Exp. Hematol. 11: 490-498; Scher, W., Scher, B. M., and Waxman, S. (1982) Biochem. & Biophys. Res. Comm. 109: 348-354); F) Tumor promoters (Huberman, E. and Callaham, M. F. (1979) Proc. Natl. Acad. Sci (USA) 76: 1293-1297; Lottem J. and Sachs, L. (1979) Proc. Natl. Acad. Sci (USA) 76: 5158-5162); and G) Inhibitors of DNA or RNA synthesis (Schwartz, E. L. and Sartorelli, A. C. (1982) Cancer Res. 42: 2651-2655, Terada, M., Epner, E., Nudel, U., Salmon, J., Fibach, E., Rifkind; R. A., and Marks, P. A. (1978) Proc. Natl. Acad. Sci (USA) 75: 2795-2799; Morin, M. J. and Sartorelli, A. C. (1984) Cancer Res. 44: 2807-2812; Schwartz, E. L., Brown, B. J., Nierenberg, M., Marsh, J. C., and Sartorelli, A. C. (1983) Cancer Res. 43: 2725-2730; Sugano, H., Furusawa, M., Kawaguchi, T., and Ikawa, Y. (1973) Bibl. Hematol. 39: 943-954; Ebert, P. S., Wars, I., and Buell, D. N. (1976) Cancer Res. 36: 1809-1813; Hayashi, M., Okabe, J., and Hozumi, M. (1979) Gann 70: 235-238).

As used herein, the term “therapeutically effective amount” is intended to qualify the combined amount of treatments in the combination therapy. The combined amount will achieve the desired biological response. In the present invention, the desired biological response is partial or total inhibition, delay or prevention of the progression of cancer including cancer metastasis; inhibition, delay or prevention of the recurrence of cancer including cancer metastasis; or the prevention of the onset or development of cancer (chemoprevention) in a mammal, for example a human.

As used herein, the terms “combination treatment”, “combination therapy”, “combined treatment,” or “combinatorial treatment”, used interchangeably, refer to a treatment of an individual with at least two different therapeutic agents. According to one aspect of the invention, the individual is treated with a first therapeutic agent, e.g., SAHA or another HDAC inhibitor as described herein. The second therapeutic agent may be another HDAC inhibitor, or may be any clinically established anti-cancer agent such as a retinoid agent as defined herein. A combinatorial treatment may include a third or even further therapeutic agent. The combination treatments may be carried out consecutively or concurrently.

A “retinoid” or “retinoid agent” (e.g., 3-methyl TTNEB; also known in the art as “Targretin” and “Bexarotene”) as used herein encompasses any synthetic, recombinant, or naturally-occurring compound that binds to one or more retinoid receptors, including any pharmaceutically acceptable salts or hydrates of such agents, and any free acids, free bases, or other free forms of such agents. Specific examples of these agents are provided herein.

As recited herein, “HDAC inhibitor” (e.g., SAHA; also known in the art as “Vorinostat”) encompasses any synthetic, recombinant, or naturally-occurring inhibitor, including any pharmaceutical salts or hydrates of such inhibitors, and any free acids, free bases, or other free forms of such inhibitors. “Hydroxamic acid derivative,” as used herein, refers to the class of histone deacetylase inhibitors that are hydroxamic acid derivatives. Specific examples of inhibitors are provided herein.

“Patient” or “subject” as the terms are used herein, refer to the recipient of the treatment. Mammalian and non-mammalian patients are included. In a specific embodiment, the patient is a mammal, such as a human, canine, murine, feline, bovine, ovine, swine, or caprine. In a particular embodiment, the patient is a human.

The terms “intermittent” or “intermittently” as used herein means stopping and starting at either regular or irregular intervals.

The term “hydrate” includes but is not limited to hemihydrate, monohydrate, dihydrate, trihydrate, and the like.

### Histone Deacetylases and Histone Deacetylase Inhibitors

Histone deacetylases (HDACs) include enzymes that catalyze the removal of acetyl groups from lysine residues in the amino terminal tails of the nucleosomal core histones. As such, HDACs together with histone acetyl transferases (HATs) regulate the acetylation status of histones. Histone acetylation affects gene expression and inhibitors of HDACs, such as the hydroxamic acid-based hybrid polar compound suberoylanilide hydroxamic acid (SAHA) induce growth arrest, differentiation, and/or apoptosis of transformed cells in vitro and inhibit tumor growth in vivo.

HDACs can be divided into three classes based on structural homology. Class I HDACs (HDACs 1, 2, 3, and 8) bear similarity to the yeast RPD3 protein, are located in the nucleus and are found in complexes associated with transcriptional co-repressors. Class II HDACs (HDACs 4, 5, 6, 7 and 9) are similar to the yeast HDA1 protein, and have both nuclear and cytoplasmic subcellular localization. Both Class I and II HDACs are inhibited by hydroxamic acid-based HDAC inhibitors, such as SAHA. Class III HDACs form a structurally distant class of NAD dependent enzymes that are related to the yeast SIR2 proteins and are not inhibited by hydroxamic acid-based HDAC inhibitors.

Histone deacetylase inhibitors or HDAC inhibitors are compounds that are capable of inhibiting the deacetylation of histones in vivo, in vitro or both. As such, HDAC inhibitors inhibit the activity of at least one histone deacetylase. As a result of inhibiting the deacetylation of at least one histone, an increase in acetylated histone occurs and accumulation of acetylated histone is a suitable biological marker for assessing the activity of HDAC inhibitors. Therefore, procedures that can assay for the accumulation of acetylated histones can be used to determine the HDAC inhibitory activity of compounds of interest. It is understood that compounds that can inhibit histone deacetylase activity can also bind to other substrates and as such can inhibit other biologically active molecules such as enzymes. It is also to be understood that the compounds of the present invention are capable of inhibiting any of the histone deacetylases set forth above, or any other histone deacetylases.

For example, in patients receiving HDAC inhibitors, the accumulation of acetylated histones in peripheral mononuclear cells as well as in tissue treated with HDAC inhibitors can be determined against a suitable control.

HDAC inhibitory activity of a particular compound can be determined in vitro using, for example, an enzymatic assay which shows inhibition of at least one histone deacetylase. Further, determination of the accumulation of acetylated histones in cells treated with a particular composition can be determinative of the HDAC inhibitory activity of a compound.

Assays for the accumulation of acetylated histones are well known in the literature. See, for example, Marks, P. A. et al., J. Natl. Cancer Inst., 92:1210-1215, 2000, Butler, L. M. et al., Cancer Res. 60:5165-5170 (2000), Richon, V. M. et al., Proc. Natl. Acad. Sci., USA, 95:3003-3007, 1998, and Yoshida, M. et al., J. Biol. Chem., 265:17174-17179, 1990.

For example, an enzymatic assay to determine the activity of an HDAC inhibitor compound can be conducted as follows. Briefly, the effect of an HDAC inhibitor compound on affinity purified human epitope-tagged (Flag) HDAC1 can be assayed by incubating the enzyme preparation in the absence of substrate on ice for about 20 minutes with the indicated amount of inhibitor compound. Substrate ([³H]acetyl-labeled murine erythroleukemia cell-derived histone) can be added and the sample can be incubated for 20 minutes at 37° C. in a total volume of 30 μL. The reaction can then be stopped and released acetate can be extracted and the amount of radioactivity release determined by scintillation counting. An alternative assay useful for determining the activity of an HDAC inhibitor compound is the “HDAC Fluorescent Activity Assay; Drug Discovery Kit-AK-500” available from BIOMOL® Research Laboratories, Inc., Plymouth Meeting, Pa.

In vivo studies can be conducted as follows. Animals, for example, mice, can be injected intraperitoneally with an HDAC inhibitor compound. Selected tissues, for example, brain, spleen, liver etc, can be isolated at predetermined times, post administration. Histones can be isolated from tissues essentially as described by Yoshida et al., J. Biol. Chem. 265:17174-17179, 1990. Equal amounts of histones (about 1 μg) can be electrophoresed on 15% SDS-polyacrylamide gels and can be transferred to Hybond-P filters (available from Amersham). Filters can be blocked with 3% milk and can be probed with a rabbit purified polyclonal anti-acetylated histone H4 antibody (αAc-H4) and anti-acetylated histone H3 antibody (αAc-H3) (Upstate Biotechnology, Inc.). Levels of acetylated histone can be visualized using a horseradish peroxidase-conjugated goat anti-rabbit antibody (1:5000) and the SuperSignal chemiluminescent substrate (Pierce). As a loading control for the histone protein, parallel gels can be run and stained with Coomassie Blue (CB).

In addition, hydroxamic acid-based HDAC inhibitors have been shown to up regulate the expression of the p21_{WAF1} gene. The p21_{WAF1} protein is induced within 2 hours of culture with HDAC inhibitors in a variety of transformed cells using standard methods. The induction of the p21_{WAF1} gene is associated with accumulation of acetylated histones in the chromatin region of this gene. Induction of p21_{WAF1} can therefore be recognized as involved in the G1 cell cycle arrest caused by HDAC inhibitors in transformed cells.

U.S. Pat. Nos. 5,369,108, 5,932,616, 5,700,811, 6,087,367 and 6,511,990, issued to some of the present inventors, disclose compounds useful for selectively inducing terminal differentiation of neoplastic cells, which compounds have two polar end groups separated by a flexible chain of methylene groups or a by a rigid phenyl group, wherein one or both of the polar end groups is a large hydrophobic group. Some of the compounds have an additional large hydrophobic group at the same end of the molecule as the first hydrophobic group which further increases differentiation activity about 100 fold in an enzymatic assay and about 50 fold in a cell differentiation assay. Methods of synthesizing the compounds used in the methods and pharmaceutical compositions of this invention are fully described the aforementioned patents, the entire contents of which are incorporated herein by reference.

Thus, the present invention includes within its broad scope compositions comprising HDAC inhibitors which are 1) hydroxamic acid derivatives; 2) Short-Chain Fatty Acids (SCFAs); 3) cyclic tetrapeptides; 4) benzamides; 5) electrophilic ketones; and/or any other class of compounds capable of inhibiting histone deacetylases, for use in inhibiting histone deacetylase, inducing terminal differentiation, cell growth arrest and/or apoptosis in neoplastic cells, and/or inducing differentiation, cell growth arrest and/or apoptosis of tumor cells in a tumor.

Non-limiting examples of such HDAC inhibitors are set forth below. It is understood that the present invention includes any salts, crystal structures, amorphous structures, hydrates, derivatives, metabolites, stereoisomers, structural isomers, and prodrugs of the HDAC inhibitors described herein.

A. Hydroxamic Acid Derivatives such as Suberoylanilide hydroxamic acid (SAHA) (Richon et al., Proc. Natl. Acad. Sci. USA 95, 3003-3007 (1998)); m-Carboxycinnamic acid bishydroxamide (CBHA) (Richon et al., supra); Pyroxamide; Trichostatin analogues such as Trichostatin A (TSA) and Trichostatin C Koghe et al. 1998. Biochem. Pharmacol. 56: 1359-1364); Salicylbishydroxamic acid (Andrews et al., International J. Parasitology 30, 761-768 (2000)); Suberoyl bishydroxamic acid (SBHA) (U.S. Pat. No. 5,608,108); Azelaic bishydroxamic acid (ABHA) (Andrews et al., supra); Azelaic-1-hydroxamate-9-anilide (AAHA) (Qiu et al., Mol. Biol. Cell 11, 2069-2083 (2000)); 6-(3-Chlorophenylureido) carpoic hydroxamic acid (3C1-UCHA); Oxamflatin [(2E)-5-[3-[(phenylsulfonyl)aminol phenyl]-pent-2-en-4-ynohydroxamic acid) (Kim et al. Oncogene, 18: 2461 2470 (1999)); A-161906, Scriptaid (Su et al. 2000 Cancer Research, 60: 3137-3142); PXD-101 (Prolifix); LAQ-824; CHAP; MW2796 (Andrews et al., supra); MW2996 (Andrews et al., supra); or any of the hydroxamic acids disclosed in U.S. Pat. Nos. 5,369,108, 5,932,616, 5,700,811, 6,087,367, and 6,511,990.

B. Cyclic Tetrapeptides such as Trapoxin A (TPX)-cyclic tetrapeptide (cyclo-(L-phenylalanyl-L-phenylalanyl-D-pipecolinyl-L-2-amino-8-oxo-9,10-epoxy decanoyl)) (Kij ima et al., J. Biol. Chem. 268, 22429-22435 (1993)); FR901228 (PK 228, depsipeptide) (Nakajima et al., Ex. Cell Res. 241, 126-133 (1998)); FR225497 cyclic tetrapeptide (H. Mori et al., PCT Application WO 00/08048 (17 Feb. 2000)); Apicidin cyclic tetrapeptide [cyclo(N—O-methyl-L-tryptophanyl-L-isoleucinyl-D-pipecolinyl-L-2-amino-8-oxodecanoyl)] (Darkin-Rattray et al., Proc. Natl. Acad. Sci. USA 93, 13143-13147 (1996)); Apicidin Ia, Apicidin Ib, Apicidin Ic, Apicidin IIa, and Apicidin IIb (P. Dulski et al., PCT Application WO 97/11366); CHAP, HC-toxin cyclic tetrapeptide (Bosch et al., Plant Cell 7, 1941-1950 (1995)); WF27082 cyclic tetrapeptide (PCT Application WO 98/48825); and Chlamydocin (Bosch et al., supra).

C. Short chain fatty acid (SCFA) derivatives such as: Sodium Butyrate (Cousens et al., J. Biol. Chem. 254, 1716-1723 (1979)); Isovalerate (McBain et al., Biochem. Pharm. 53: 1357-1368 (1997)); Valerate (McBain et al., supra); 4-Phenylbutyrate (4-PBA) (Lea and Tulsyan, Anticancer Research, 15, 879-873 (1995)); Phenylbutyrate (PB) (Wang et al., Cancer Research, 59, 2766-2799 (1999)); Propionate (McBain et al., supra); Butyramide (Lea and Tulsyan, supra); Isobutyramide (Lea and Tulsyan, supra); Phenylacetate (Lea and Tulsyan, supra); 3-Bromopropionate (Lea and Tulsyan, supra); Tributyrin (Guan et al., Cancer Research, 60, 749-755 (2000)); Valproic acid, Valproate, and Pivanex™.

D. Benzamide derivatives such as CI-994; MS-275 [N-(2-aminophenyl)-4-[N-(pyridin-3-yl methoxycarbonyl)aminomethyl]benzamide] (Saito et al., Proc. Natl. Acad. Sci. USA 96, 4592-4597 (1999)); and 3′-amino derivative of MS-275 (Saito et al., supra).

E. Electrophilic ketone derivatives such as Trifluoromethyl ketones (Frey et al, Bioorganic & Med. Chem. Lett. (2002), 12, 3443-3447; U.S. Pat. No. 6,511,990) and α-keto amides such as N-methyl-α-ketoamides.

F. Other HDAC Inhibitors such as natural products, psammaplins, and Depudecin (Kwon et al. 1998. PNAS 95: 3356-3361).

Hydroxamic acid based HDAC inhibitors include suberoylanilide hydroxamic acid (SAHA), m-carboxycinnamic acid bishydroxamate (CBHA) and pyroxamide. SAHA has been shown to bind directly in the catalytic pocket of the histone deacetylase enzyme. SAHA induces cell cycle arrest, differentiation, and/or apoptosis of transformed cells in culture and inhibits tumor growth in rodents. SAHA is effective at inducing these effects in both solid tumors and hematological cancers. It has been shown that SAHA is effective at inhibiting tumor growth in animals with no toxicity to the animal. The SAHA-induced inhibition of tumor growth is associated with an accumulation of acetylated histones in the tumor. SAHA is effective at inhibiting the development and continued growth of carcinogen-induced (N-methylnitrosourea) mammary tumors in rats. SAHA was administered to the rats in their diet over the 130 days of the study. Thus, SAHA is a nontoxic, orally active antitumor agent whose mechanism of action involves the inhibition of histone deacetylase activity.

HDAC inhibitors include those disclosed in U.S. Pat. Nos. 5,369,108, 5,932,616, 5,700,811, 6,087,367, and 6,511,990, issued to some of the present inventors disclose compounds, the entire contents of which are incorporated herein by reference, non-limiting examples of which are set forth below:

Specific HDAC inhibitors include suberoylanilide hydroxamic acid (SAHA; N-Hydroxy-N′-phenyl octanediamide), which is represented by the following structural formula:

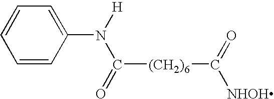

Other examples of such compounds and other HDAC inhibitors can be found in U.S. Pat. No. 5,369,108, issued on Nov. 29, 1994, U.S. Pat. No. 5,700,811, issued on Dec. 23, 1997, U.S. Pat. No. 5,773,474, issued on Jun. 30, 1998, U.S. Pat. No. 5,932,616, issued on Aug. 3, 1999 and U.S. Pat. No. 6,511,990, issued Jan. 28, 2003, all to Breslow et al.; U.S. Pat. No. 5,055,608, issued on Oct. 8, 1991, U.S. Pat. No. 5,175,191, issued on Dec. 29, 1992 and U.S. Pat. No. 5,608,108, issued on Mar. 4, 1997, all to Marks et al.; as well as Yoshida, M., et al., Bioassays 17, 423-430 (1995); Saito, A., et al., PNAS USA 96, 4592-4597, (1999); Furamai R. et al., PNAS USA 98 (1), 87-92 (2001); Komatsu, Y., et al., Cancer Res. 61(11), 4459-4466 (2001); Su, G. H., et al., Cancer Res. 60, 3137-3142 (2000); Lee, B. I. et al., Cancer Res. 61(3), 931-934; Suzuki, T., et al., J. Med. Chem. 42(15), 3001-3003 (1999); published PCT Application WO 01/18171 published on Mar. 15, 2001 to Sloan-Kettering Institute for Cancer Research and The Trustees of Columbia University; published PCT Application WO 02/246144 to Hoffmann-La Roche; published PCT Application WO 02/22577 to Novartis; published PCT Application WO 02/30879 to Prolifix; published PCT Applications WO 01/38322 (published May 31, 2001), WO 01/70675 (published on Sep. 27, 2001) and WO 00/71703 (published on Nov. 30, 2000) all to Methylgene, Inc.; published PCT Application WO 00/21979 published on Oct. 8, 1999 to Fujisawa Pharmaceutical Co., Ltd.; published PCT Application WO 98/40080 published on Mar. 11, 1998 to Beacon Laboratories, L.L.C.; and Curtin M. (Current patent status of HDAC inhibitors Expert Opin. Ther. Patents (2002) 12(9): 1375-1384 and references cited therein).

SAHA or any of the other HDACs can be synthesized according to the methods outlined in the Experimental Details Section, or according to the method set forth in U.S. Pat. Nos. 5,369,108, 5,700,811, 5,932,616 and 6,511,990, the contents of which are incorporated by reference in their entirety, or according to any other method known to a person skilled in the art.

Specific non-limiting examples of HDAC inhibitors are provided in Table 1 below. It should be noted that the present invention encompasses any compounds which are structurally similar to the compounds represented below, and which are capable of inhibiting histone deacetylases.

TABLE 1
HDAC inhibitors

| Name | Structure |
|---|---|
| MS-275 | 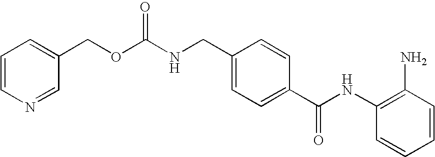 |
| DEPSIPEPTIDE | 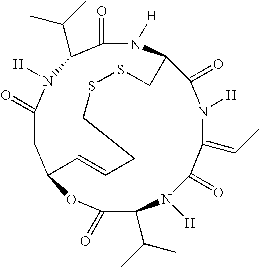 |
| CI-994 | 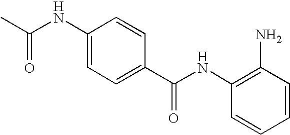 |
| Apicidin | 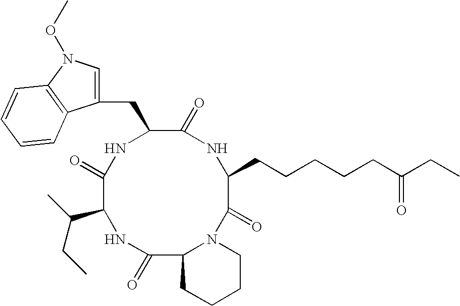 |
| A-161906 | 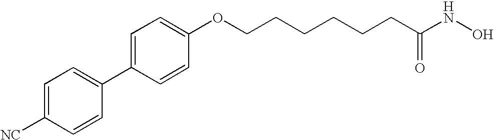 |
| Scriptaid | 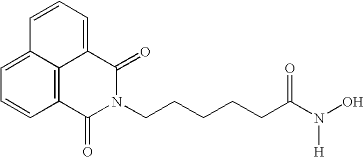 |
| PXD-101 | 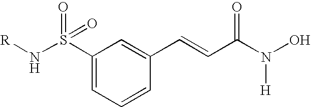 |
| CHAP | 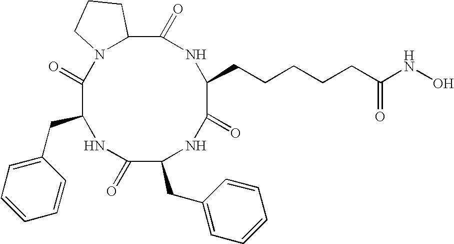 |
| LAQ-824 | 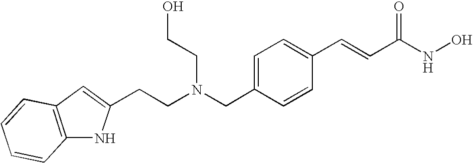 |
| Butyric Acid | 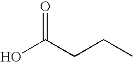 |
| Depudecin | 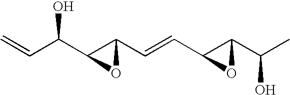 |
| Oxamflatin | 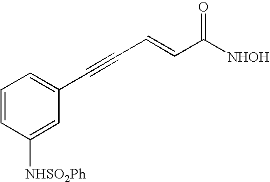 |
| Trichostatin C | 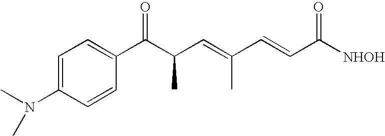 |

### Retinoids and Other Therapies

Recent developments have introduced, in addition to the traditional cytotoxic and hormonal therapies used to treat cancer, additional therapies for the treatment of cancer. For example, many forms of gene therapy are undergoing preclinical or clinical trials. In addition, approaches are currently under development that are based on the inhibition of tumor vascularization (angiogenesis). The aim of this concept is to cut off the tumor from nutrition and oxygen supply provided by a newly built tumor vascular system. In addition, cancer therapy is also being attempted by the induction of terminal differentiation of the neoplastic cells. Suitable differentiation agents include the compounds disclosed in any one or more of the following references, the contents of which are incorporated by reference herein.

A) Polar compounds (Marks et al. (1987); , Friend, C., Scher, W., Holland, J. W., and Sato, T. (1971) Proc. Natl. Acad. Sci (USA) 68: 378-382; Tanaka, M., Levy, J., Terada, M., Breslow, R., Rifkind, R. A., and Marks, P. A. (1975) Proc. Natl. Acad. Sci (USA) 72: 1003-1006; Reuben, R. C., Wife, R. L., Breslow, R., Rifkind, R. A., and Marks, P. A. (1976) Proc. Natl. Acad. Sci (USA) 73: 862-866); B) Derivatives of vitamin D and retinoic acid (Abe, E., Miyaura, C., Sakagami, H., Takeda, M., Konno, K., Yamazaki, T., Yoshika, S., and Suda, T. (1981) Proc. Nail. Acad. Sci (USA) 78: 4990-4994; Schwartz, E. L., Snoddy, J. R., Kreutter, D., Rasmussen, H., and Sartorelli, A. C. (1983) Proc. Am. Assoc. Cancer Res. 24: 18; Tanenaga, K., Hozumi, M., and Sakagami, Y. (1980) Cancer Res. 40: 914-919); C) Steroid hormones (Lotem, J. and Sachs, L. (1975) Int. J. Cancer 15: 731-740); D) Growth factors (Sachs, L. (1978) Nature (Lond) 274: 535, Metcalf, D. (1985) Science, 229: 16-22); E) Proteases (Scher, W., Scher, B. M., and Waxman, S. (1983) Exp. Hematol. 11: 490-498; Scher, W., Scher, B. M., and Waxman, S. (1982) Biochem. & Biophys. Res. Comm. 109: 348-354); F) Tumor promoters (Huberman, E. and Callaham, M. F. (1979) Proc. Natl. Acad. Sci (USA) 76: 1293-1297; Lottem, J. and Sachs, L. (1979) Proc. Natl. Acad. Sci (USA) 76: 5158-5162); and G) Inhibitors of DNA or RNA synthesis (Schwartz, E. L. and Sartorelli, A. C. (1982) Cancer Res. 42: 2651-2655, Terada, M., Epner, E., Nudel, U., Salmon, J., Fibach, E., Rifkind, R. A., and Marks, P. A. (1978) Proc. Natl. Acad. Sci (USA) 75: 2795-2799; Morin, M. J. and Sartorelli, A. C. (1984) Cancer Res. 44: 2807-2812; Schwartz, E. L., Brown, B. J., Nierenberg, M., Marsh, J. C., and Sartorelli, A. C. (1983) Cancer Res. 43: 2725-2730; Sugano, H., Furusawa, M., Kawaguchi, T., and Ikawa, Y. (1973) Bibl. Hematol. 39: 943-954; Ebert, P. S., Wars, I., and Buell, D. N. (1976) Cancer Res. 36: 1809-1813; Hayashi, M., Okabe, J., and Hozumi, M. (1979) Gann 70: 235-238),

Retinoids or retinoid agents for use with the invention include all natural, recombinant, and synthetic derivatives or mimetics of vitamin A, for example, retinyl palmitate, retinoyl-beta-glucuronide (vitamin A1 beta-glucuronide), retinyl phosphate (vitamin A1 phosphate), retinyl esters, 4-oxoretinol, 4-oxoretinaldehyde, 3-dehydroretinol (vitamin A2), 11-cis-retinal (11-cis-retinaldehyde, 11-cis or neo b vitamin A1 aldehyde), 5,6-epoxyretinol (5,6-epoxy vitamin A1 alcohol), anhydroretinol (anhydro vitamin A1) and 4-ketoretinol (4-keto-vitamin A1 alcohol), all-trans retinoic acid (ATRA; Tretinoin; vitamin A acid; 3,7-dimethyl-9-(2,6,6,-trimethyl-1-cyclohenen-1-yl)-2,4,6,8-nonatetraenoic acid [CAS No. 302-79-4]), lipid formulations of all-trans retinoic acid (e.g., ATRA-IV), 9-cis retinoic acid (9-cis-RA; Alitretinoin; Panretin©(c; LGD1057), (e)-4-[2-(5,6,7,8-tetrahydro-2-naphthalenyl)-1-propenyl]-benzoic acid, 3-methyl-(E)-4-[2-(5,6,7,8-tetrahydro-2-naphthalenyl)-1-propenyl]-benzoic acid, Fenretinide (N-(4-hydroxyphenyl)retinamide; 4-HPR), Etretinate (2,4,6,8-nonatetraenoic acid), Acitretin (Ro 10-1670), Tazarotene (ethyl 6-[2-(4,4-dimethylthiochroman-6-yl)-ethynyl]nicotinate), Tocoretinate (9-cis-tretinoin tocoferil), Adapalene (6-[3-(1-adamantyl)-4-methoxyphenyl]-2-naphthoic acid), Motretinide (trimethylmethoxyphenyl-N-ethyl retinamide), and retinaldehyde.

Also included as retinoids are retinoid related molecules such as CD437 (also called 6-[3-(1-adamantyl)-4-hydroxphenyl]-2-naphthalene carboxylic acid and AHPN), CD2325, ST1926 ([E-3-(4′-hydroxy-3′-adamantylbiphenyl-4-yl)acrylic acid), ST1878 (methyl 2-[3-[2-[3-(2-methoxy-1,1-dimethyl-2-oxoethoxy)pheno-xy]ethoxy]phenoxy]isobutyrate), ST2307, ST1898, ST2306, ST2474, MM11453, MM002 (3-C1-AHPC), MX2870-1, MX3350-1, MX84, and MX90-1 (Garattini et al., 2004, Curr. Pharmaceut. Design 10:433-448; Garattini and Terao, 2004, J. Chemother. 16:70-73). Included for use with the invention are retinoid agents that bind to one or more RXR. Also included are retinoid agents that bind to one or more RXR and do not bind to one or more RAR (i.e., selective binding to RXR; rexinoids), e.g., docosahexanoic acid (DHA), phytanic acid, methoprene acid, LG100268 (LG268), LG100324, LGD1057, SR11203, SR11217, SR11234, SR11236, SR11246, AGN194204 (see, e.g., Simeone and Tari, 2004, Cell Mol. Life. Sci. 61:1475-1484; Rigas and Dragnev, 2005, The Oncologist 10:22-33; Ahuja et al., 2001, Mol. Pharmacol. 59:765-773; Gorgun and Foss, 2002, Blood 100:1399-1403; Bischoff et al., 1999, J. Natl. Cancer Inst. 91:2118-2123; Sun et al., 1999, Clin. Cancer Res. 5:431-437; Crow and Chandraratna, 2004, Breast Cancer Res. 6:R546-R555). Further included are derivatives of 9-cis-RA. Additionally included are 3-methyl TTNEB and related agents, e.g., Targreting®; Bexarotene; LGD1069; 4-[1-(5,6,7,8-tetrahydro-3,5,5,8,8-pentamethyl-2-naphthalenyl)ethenyl]benzoic acid, as represented by the structure:

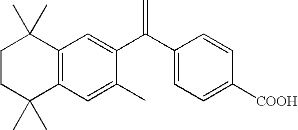

or a pharmaceutically acceptable salt or hydrate thereof.

### Stereochemistry

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and I or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture.

Many of the compounds described herein can have one or more chiral centers and therefore can exist in different enantiomeric forms. If desired, a chiral carbon can be designated with an asterisk (*). When bonds to the chiral carbon are depicted as straight lines in the formulas of the invention, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the HDAC inhibitors of the present invention contain one chiral center, the compounds exist in two enantiomeric forms and the present invention includes both enantiomers and mixtures of enantiomers, such as the specific 50:50 mixture referred to as a racemic mixtures. The enantiomers can be resolved by methods known to those skilled in the art, for example by formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form.

Alternatively, specific enantiomers may be synthesized by asymmetric synthesis using optically active reagents, substrates, catalysts or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. For example, the enantiomeric excess can be about 60% or more, such as about 70% or more, for example about 80% or more, such as about 90% or more. In a particular embodiment when a specific absolute configuration is designated, the enantiomeric excess of depicted compounds is at least about 90%. In a more particular embodiment, the enantiomeric excess of the compounds is at least about 95%, such as at least about 97.5%, for example, at least 99% enantiomeric excess.

When a compound of the present invention has two or more chiral carbons it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers which are not mirror-images (e.g., (S,S) and (R,S)) are diastereomers. The diastereoisomeric pairs may be separated by methods known to those skilled in the art, for example chromatography or crystallization and the individual enantiorners within each pair may be separated as described above. The present invention includes each diastereoisomer of such compounds and mixtures thereof.

As used herein, “a,” an” and “the” include singular and plural referents unless the context clearly dictates otherwise. Thus, for example, reference to “an active agent” or “a pharmacologically active agent” includes a single active agent as well a two or more different active agents in combination, reference to “a carrier” includes mixtures of two or more carriers as well as a single carrier, and the like.

This invention is also intended to encompass pro-drugs of the HDAC inhibitors disclosed herein. A prodrug of any of the compounds can be made using well known pharmacological techniques.

This invention, in addition to the above listed compounds, is intended to encompass the use of homologs and analogs of such compounds. In this context, homologs are molecules having substantial structural similarities to the above-described compounds and analogs are molecules having substantial biological similarities regardless of structural similarities.

### Alkylating Agents

Examples of alkylating agents include, but are not limited to, bischloroethylamines (nitrogen mustards, e.g., Chlorambucil, Cyclophosphamide, Ifosfamide, Mechlorethamine, Melphalan, uracil mustard), aziridines (e.g., Thiotepa), alkyl alkone sulfonates (e.g., Busulfan), nitrosoureas (e.g., Carmustine, Lomustine, Streptozocin), nonclassic alkylating agents (Altretamine, Dacarbazine, and Procarbazine), platinum compounds (Carboplastin and Cisplatin). These compounds react with phosphate, amino, hydroxyl, sulfihydryl, carboxyl, and imidazole groups.

Under physiological conditions, these drugs ionize and produce positively charged ion that attach to susceptible nucleic acids and proteins, leading to cell cycle arrest and/or cell death. The alkylating agents are cell cycle phasenonspecific agents because they exert their activity independently of the specific phase of the cell cycle. The nitrogen mustards and alkyl alkone sulfonates are most effective against cells in the G1 or M phase. Nitrosoureas, nitrogen mustards, and aziridines impair progression from the G1 and S phases to the M phases. Chabner and Collins eds. (1990) “Cancer Chemotherapy: Principles and Practice”, Philadelphia: J B Lippincott.

The alkylating agents are active against wide variety of neoplastic diseases, with significant activity in the treatment of leukemias and lymphomas as well as solid tumors. Clinically this group of drugs is routinely used in the treatment of acute and chronic leukemias; Hodgkin's disease; non-Hodgkin's lymphoma; multiple myeloma; primary brain tumors; carcinomas of the breast, ovaries, testes, lungs, bladder, cervix, head and neck, and malignant melanoma.

### Antibiotic Agents

Antibiotics (e.g., cytotoxic antibiotics) act by directly inhibiting DNA or RNA synthesis and are effective throughout the cell cycle. Examples of antibiotic agents include anthracyclines (e.g., Doxorubicin, Daunorubicin, Epirubicin, Idarubicin, and Anthracenedione), Mitomycin C, Bleomycin, Dactinomycin, Plicatomycin. These antibiotic agents interfere with cell growth by targeting different cellular components. For example, anthracyclines are generally believed to interfere with the action of DNA topoisomerase II in the regions of transcriptionally active DNA, which leads to DNA strand scissions.

Bleomycin is generally believed to chelate iron and forms an activated complex, which then binds to bases of DNA, causing strand scissions and cell death.

The antibiotic agents have been used as therapeutics across a range of neoplastic diseases, including carcinomas of the breast, lung, stomach and thyroids, lymphomas, myelogenous leukemias, myelomas, and sarcomas.

### Antimetabolic Agents

Antimetabolic agents (i.e., antimetabolites) are a group of drugs that interfere with metabolic processes vital to the physiology and proliferation of cancer cells. Actively proliferating cancer cells require continuous synthesis of large quantities of nucleic acids, proteins, lipids, and other vital cellular constituents.

Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Antimitotic agents are included in this group. Examples of antimetabolic agents include, but are not limited to, Fluorouracil (5-FU), Floxuridine (5-FUdR), Methotrexate, Leucovorin, Hydroxyurea, Thioguanine (6-TG), Mercaptopurine (6-MP), Cytarabine, Pentostatin, Fludarabine Phosphate, Cladribine (2-CDA), Asparaginase, and Gemcitabine.

Antimetabolic agents have widely used to treat several common forms of cancer including carcinomas of colon, rectum, breast, liver, stomach and pancreas, malignant melanoma, acute and chronic leukemia and hair cell leukemia

### Hormonal Agents

The hormonal agents are a group of drugs that regulate the growth and development of their target organs. Most of the hormonal agents are sex steroids and their derivatives and analogs thereof, such as estrogens, progestogens, anti-estrogens, androgens, anti-androgens and progestins. These hormonal agents may serve as antagonists of receptors for the sex steroids to down regulate receptor expression and transcription of vital genes. Examples of such hormonal agents are synthetic estrogens (e.g., Diethylstibestrol), antiestrogens (e.g., Tamoxifen, Toremifene, Fluoxymesterol, and Raloxifene), antiandrogens (e.g., Bicalutamide, Nilutamide, and Flutamide), aromatase inhibitors (e.g., Aminoglutethimide, Anastrozole, and Tetrazole), luteinizing hormone release hormone (LHRH) analogues, Ketoconazole, Goserelin Acetate, Leuprolide, Megestrol Acetate, and Mifepristone.

Hormonal agents are used to treat breast cancer, prostate cancer, melanoma, and meningioma. Because the major action of hormones is mediated through steroid receptors, 60% receptor-positive breast cancer responded to first-line hormonal therapy; and less than 10% of receptor-negative tumors responded. The main side effect associated with hormonal agents is flare. The frequent manifestations are an abrupt increase of bony pain, erythema around skin lesions, and induced hypercalcemia.

Specifically, progestogens are used to treat endometrial cancers, since these cancers occur in women that are exposed to high levels of oestrogen unopposed by progestogen.

Antiandrogens are used primarily for the treatment of prostate cancer, which is hormone dependent. They are used to decrease levels of testosterone, and thereby inhibit growth of the tumor.

Hormonal treatment of breast cancer involves reducing the level of oestrogen-dependent activation of oestrogen receptors in neoplastic breast cells. Anti-oestrogens act by binding to oestrogen receptors and prevent the recruitment of coactivators, thus inhibiting the oestrogen signal.

LHRH analogues are used in the treatment of prostate cancer to decrease levels of testosterone and so decrease the growth of the tumor.

Aromatase inhibitors act by inhibiting the enzyme required for hormone synthesis. In post-menopausal women, the main source of oestrogen is through the conversion of androstenedione by aromatase.

### Plant-Derived Agents

Plant-derived agents are a group of drugs that are derived from plants or modified based on the molecular structure of the agents. They inhibit cell replication by preventing the assembly of the cell's components that are essential to cell division.

Examples of plant derived agents include vinca alkaloids (e.g., Vincristine, Vinblastine, Vindesine, Vinzolidine, and Vinorelbine), podophyllotoxins (e.g., Etoposide (VP-16) and Teniposide (VM-26)), and taxanes (e.g., Paclitaxel and Docetaxel). These plant-derived agents generally act as antimitotic agents that bind to tubulin and inhibit mitosis. Podophyllotoxins such as etoposide are believed to interfere with DNA synthesis by interacting with topoisomerase II, leading to DNA strand scission.

Plant-derived agents are used to treat many forms of cancer. For example, vincristine is used in the treatment of the leukemias, Hodgkin's and non-Hodgkin's lymphoma, and the childhood tumors neuroblastoma, rhabdomyosarcoma, and Wilms' tumor. Vinblastine is used against the lymphomas, testicular cancer, renal cell carcinoma, mycosis fungoides, and Kaposi's sarcoma. Docetaxel has shown promising activity against advanced breast cancer, non-small cell lung cancer (NSCLC), and ovarian cancer.

Etoposide is active against a wide range of neoplasms, of which small cell lung cancer, testicular cancer, and NSCLC are most responsive.

### Biologic Agents

Biologic agents are a group of biomolecules that elicit cancer/tumor regression when used alone or in combination with chemotherapy and/or radiotherapy. Examples of biologic agents include immunomodulating proteins such as cytokines, monoclonal antibodies against tumor antigens, tumor suppressor genes, and cancer vaccines.

Cytokines possess profound immunomodulatory activity. Some cytokines such as interleukin-2 (IL-2, Aldesleukin) and interferon-a(IFN-a) demonstrated antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and nonself.

Interferon-α includes more than 23 related subtypes with overlapping activities. IFN-α has demonstrated activity against many solid and hematologic malignancies, the later appearing to be particularly sensitive.

Examples of interferons include interferon-α, interferon-β (fibroblast interferon) and interferon-γ (fibroblast interferon). Examples of other cytokines include erythropoietin (Epoietin-α), granulocyte-CSF (Filgrastin), and granulocyte, macrophage-CSF (Sargramostim). Other immuno-modulating agents other than cytokines include bacillus Calmette-Guerin, levamisole, and octreotide, a long-acting octapeptide that mimics the effects of the naturally occurring hormone somatostatin.

Furthermore, the anti-cancer treatment can comprise treatment by immunotherapy with antibodies and reagents used in tumor vaccination approaches. The primary drugs in this therapy class are antibodies, alone or carrying e.g. toxins or chemotherapeutics/cytotoxics to cancer cells. Monoclonal antibodies against tumor antigens are antibodies elicited against antigens expressed by tumors, particularly tumor-specific antigens. For example, monoclonal antibody HERCEPTIN® (Trastuzumab) is raised against human epidermal growth factor receptor2 (HER2) that is overexpressed in some breast tumors including metastatic breast cancer. Overexpression of HER2 protein is associated with more aggressive disease and poorer prognosis in the clinic. HERCEPTIN® is used as a single agent for the treatment of patients with metastatic breast cancer whose tumors over express the HER2 protein.

Another example of monoclonal antibodies against tumor antigens is RITUXAN® (Rituximab) that is raised against CD20 on lymphoma cells and selectively deplete normal and malignant CD20+ pre-B and mature B cells.

RITUXAN is used as single agent for the treatment of patients with relapsed or refractory low-grade or follicular, CD20+, B cell non-Hodgkin's lymphoma. MYELOTARG® (Gemtuzumab Ozogamicin) and CAMPATH® (Alemtuzumab) are further examples of monoclonal antibodies against tumor antigens that may be used.

Endostatin is a cleavage product of plasminogen used to target angiogenesis.

Tumor suppressor genes are genes that function to inhibit the cell growth and division cycles, thus preventing the development of neoplasia. Mutations in tumor suppressor genes cause the cell to ignore one or more of the components of the network of inhibitory signals, overcoming the cell cycle checkpoints and resulting in a higher rate of controlled cell growth-cancer. Examples of the tumor suppressor genes include Duc-4, NF-1, NF-2, RB, p53, WT1, BRCA1, and BRCA2.

DPC4 is involved in pancreatic cancer and participates in a cytoplasmic pathway that inhibits cell division. NF-1 codes for a protein that inhibits Ras, a cytoplasmic inhibitory protein. NF-1 is involved in neurofibroma and pheochromocytomas of the nervous system and myeloid leukemia. NF-2 encodes a nuclear protein that is involved in meningioma, schwanoma, and ependymoma of the nervous system. RB codes for the pRB protein, a nuclear protein that is a major inhibitor of cell cycle. RB is involved in retinoblastoma as well as bone, bladder, small cell lung and breast cancer. P53 codes for p53 protein that regulates cell division and can induce apoptosis. Mutation and/or inaction of p53 is found in a wide range of cancers. WTI is involved in Wilms' tumor of the kidneys. BRCA1 is involved in breast and ovarian cancer, and BRCA2 is involved in breast cancer. The tumor suppressor gene can be transferred into the tumor cells where it exerts its tumor suppressing functions.

Cancer vaccines are a group of agents that induce the body's specific immune response to tumors. Most of cancer vaccines under research and development and clinical trials are tumor-associated antigens (TAAs). TAAs are structures (i.e., proteins, enzymes, or carbohydrates) that are present on tumor cells and relatively absent or diminished on normal cells. By virtue of being fairly unique to the tumor cell, TAAs provide targets for the immune system to recognize and cause their destruction. Examples of TAAs include gangliosides (GM2), prostate specific antigen (PSA), α-fetoprotein (AFP), carcinoembryonic antigen (CEA) (produced by colon cancers and other adenocarcinomas, e.g., breast, lung, gastric, and pancreatic cancers), melanoma-associated antigens (MART-1, gap100, MAGE 1,3 tyrosinase), papillomavirus E6 and E7 fragments, whole cells or portions/lysates of autologous tumor cells and allogeneic tumor cells.

The use of all of these approaches in combination with HDAC inhibitors, e.g. SAHA, and retinoid agents, e.g., Targretin, is within the scope of the present invention.

### Administration of the HDAC Inhibitor

### Routes of Administration

The HDAC inhibitor (e.g. SAHA) and the retinoid agent (e.g. Targretin) and optionally another anti-cancer agent, can be administered by any known administration method known to a person skilled in the art. Examples of routes of administration include but are not limited to oral, parenteral, intraperitoneal, intravenous, intraarterial, transdermal, topical, sublingual, intramuscular, rectal, transbuccal, intranasal, liposomal, via inhalation, vaginal, intraoccular, via local delivery by catheter or stent, subcutaneous, intraadiposal, intraarticular, intrathecal, or in a slow release dosage form. SAHA or any one of the HDAC inhibitors can be administered in accordance with any dose and dosing schedule that, together with the effect of the anti-cancer agent such as a retinoid agent like Targretin and optionally another anti-cancer agent, achieves a dose effective to treat disease.

Of course, the route of administration of SAHA or any one of the other HDAC inhibitors can be independent of the route of administration of the retinoid agent and optional anti-cancer agent. A particular route of administration for SAHA is oral administration. Thus, in accordance with this embodiment, SAHA is administered orally, and the second agent (anti-cancer agent such as a retinoid agent, e.g. Targretin) and optional third agent can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

As examples, the HDAC inhibitors of the invention, as well as the retinoid agents and optional additional anti-cancer agents, can be administered in such oral forms as tablets, capsules (each of which includes sustained release or timed release formulations), pills, powders, granules, elixirs, tinctures, suspensions, syrups, and emulsions. Likewise, the HDAC inhibitors, retinoid agents, and optional additional anti-cancer agent can be administered by intravenous (e.g., bolus or infusion), intraperitoneal, subcutaneous, intramuscular, or other routes using forms well known to those of ordinary skill in the pharmaceutical arts. A particular route of administration of the HDAC inhibitor is oral administration.

The HDAC inhibitors can also be administered in the form of a depot injection or implant preparation, which may be formulated in such a manner as to permit a sustained release of one or more active ingredients. The active ingredient(s) can be compressed into pellets or small cylinders and implanted subcutaneously or intramuscularly as depot injections or implants. Implants may employ inert materials such as biodegradable polymers or synthetic silicones, for example, Silastic, silicone rubber or other polymers manufactured by the Dow-Corning Corporation.

The HDAC inhibitor, retinoid agent, and optional additional anti-cancer agent can also be administered in the form of liposome delivery systems, such as small unilamellar vesicles, large unilamellar vesicles and multilamellar vesicles. Liposomes can be formed from a variety of phospholipids, such as cholesterol, stearylamine, or phosphatidylcholines. Liposomal preparations of retinoid agents may also be used in the methods of the invention. Liposome versions of retinoid agents may be used to increase tolerance to the agents. For example, liposomal tretinoins such as liposomal ATRA or ATRA-IV may be used. The HDAC inhibitor, retinoid agent, and optional additional anti-cancer agent can be contained together in the liposome preparation, or can each be contained in separate liposome preparations.

The HDAC inhibitors, retinoid agent, and optional additional anti-cancer agent can also be delivered by the use of monoclonal antibodies as individual carriers to which the compound molecules are coupled.

The HDAC inhibitors, retinoid agents, and optional additional anti-cancer agents can also be prepared with soluble polymers as targetable drug carriers. Such polymers can include polyvinlypyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the HDAC inhibitors, retinoid agents, and optional additional anti-cancer agents can be prepared with biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

In one embodiment, the HDAC inhibitor, e.g. SAHA, is administered orally in a gelatin capsule, which can comprise excipients such as microcrystalline cellulose, croscarmellose sodium and magnesium stearate. A further embodiment includes 200 mg of solid SAHA with 89.5 mg of microcrystalline cellulose, 9 mg of sodium croscamiellose, and 1.5 mg of magnesium stearate contained in a gelatin capsule.

### Dosages and Dosage Schedules

The dosage regimen utilizing the HDAC inhibitors, retinoid agents, and optional additional anti-cancer agents can be selected in accordance with a variety of factors including type, species, age, weight, sex and the type of disease being treated; the severity (i.e., stage) of the disease to be treated; the route of administration; the renal and hepatic function of the patient; and the particular compound or salt thereof employed. A dosage regiment can be used, for example, to prevent, inhibit (fully or partially), or arrest the progress of the disease.

In accordance with the invention, an HDAC inhibitor (e.g., SAHA or a pharmaceutically acceptable salt or hydrate thereof), retinoid agents (e.g. Targretin or a pharmaceutically acceptable salt or hydrate thereof), and optional additional anti-cancer agents can be administered by continuous or intermittent dosages. For example, intermittent administration of an HDAC inhibitor in combination with retinoid agents, and optional additional anti-cancer agents may comprise administration one to six days per week or it may mean administration in cycles (e.g. daily administration for two to eight consecutive weeks, then a rest period with no administration for up to one week) or it may mean administration on alternate days. The compositions may be administered in cycles, with rest periods in between the cycles (e.g. treatment for two to eight weeks with a rest period of up to a week between treatments).

For example, SAHA or any one of the HDAC inhibitors can be administered in a total daily dose of up to 800 mg. The HDAC inhibitor can be administered once daily (QD), or divided into multiple daily doses such as twice daily (BID), and three times daily (TID). The HDAC inhibitor can be administered at a total daily dosage of up to 800 mg, e.g., 200 mg, 300 mg, 400 mg, 600 mg, or 800 mg, which can be administered in one daily dose or can be divided into multiple daily doses as described above. In specific aspects, the administration is oral.

In one embodiment, the composition is administered once daily at a dose of about 200-600 mg. In another embodiment, the composition is administered twice daily at a dose of about 200-400 mg. In another embodiment, the composition is administered twice daily at a dose of about 200-400 mg intermittently, for example three, four or five days per week. In one embodiment, the daily dose is 200 mg which can be administered once-daily, twice-daily or three-times daily. In one embodiment, the daily dose is 300 mg which can be administered once-daily, twice-daily or three-times daily. In one embodiment, the daily dose is 400 mg which can be administered once-daily, twice-daily or three-times daily.

SAHA or any one of the HDAC inhibitors can be administered in accordance with any dose and dosing schedule that, together with the effect of retinoid agents, and optional additional anti-cancer agents, achieves a dose effective to treat cancer. The HDAC inhibitors, retinoid agents, and optional additional anti-cancer agents can be administered in a total daily dose that may vary from patient to patient, and may be administered at varying dosage schedules. For example, SAHA or any of the HDAC inhibitors can be administered to the patient at a total daily dosage of between 25-4000 mg/m². In particular, SAHA or any one of the HDAC inhibitors can be administered in a total daily dose of up to 800 mg, especially by oral administration, once, twice or three times daily, continuously (every day) or intermittently (e.g., 3-5 days a week). In addition, the administration can be continuous, i.e., every day, or intermittently.

A particular treatment protocol comprises continuous administration (i.e., every day), once, twice or three times daily at a total daily dose in the range of about 200 mg to about 600 mg. Another treatment protocol comprises intermittent administration of between three to five days a week, once, twice or three times daily at a total daily dose in the range of about 200 mg to about 600 mg.

In one particular embodiment, the HDAC inhibitor is administered continuously once daily at a dose of 400 mg or twice daily at a dose of 200 mg.

In another particular embodiment, the HDAC inhibitor is administered intermittently three days a week, once daily at a dose of 400 mg or twice daily at a dose of 200 mg.

In another particular embodiment, the HDAC inhibitor is administered intermittently four days a week, once daily at a dose of 400 mg or twice daily at a dose of 200 mg.

In another particular embodiment, the HDAC inhibitor is administered intermittently five days a week, once daily at a dose of 400 mg or twice daily at a dose of 200 mg.

In one particular embodiment, the HDAC inhibitor is administered continuously once daily at a dose of 600 mg, twice daily at a dose of 300 mg, or three times daily at a dose of 200 mg.

In another particular embodiment, the HDAC inhibitor is administered intermittently three days a week, once daily at a dose of 600 mg, twice daily at a dose of 300 mg, or three times daily at a dose of 200 mg.

In another particular embodiment, the HDAC inhibitor is administered intermittently four days a week, once daily at a dose of 600 mg, twice daily at a dose of 300 mg, or three times daily at a dose of 200 mg.

In another particular embodiment, the HDAC inhibitor is administered intermittently five days a week, once daily at a dose of 600 mg, twice daily at a dose of 300 mg, or three times daily at a dose of 200 mg.

In one embodiment, the composition is administered continuously (i.e., daily) or intermittently (e.g., at least 3 days per week) with a once daily dose of about 300 mg, about 400 mg, about 500 mg, about 600 mg, about 700 mg, or about 800 mg.

In another embodiment, the composition is administered once daily at a dose of about 300 mg, about 400 mg, about 500 mg, about 600 mg, about 700 mg, or about 800 mg for at least one period of 7 out of 21 days (e.g., 7 consecutive days or Days 1-7 in a 21 day cycle).

In another embodiment, the composition is administered once daily at a dose of about 400 mg, about 500 mg, or about 600 mg for at least one period of 14 out of 21 days (e.g., 14 consecutive days or Days 1-14 in a 21 day cycle).

In another embodiment, the composition is administered once daily at a dose of about 300 mg or about 400 mg for at least one period of 14 out of 28 days (e.g., 14 consecutive days or Days 1-14 of a 28 day cycle).

In another embodiment, the composition is administered once daily at a dose of about 400 mg, for example, for at least one period of 21 out of 28 days (e.g., 21 consecutive days or Days 1-21 in a 28 day cycle).

In another embodiment, the composition is administered continuously (i.e., daily) or intermittently (e.g., at least 3 days per week) with a twice daily dose of about 200 mg, about 250 mg, about 300 mg, or about 400 mg.

In another embodiment, the composition is administered twice daily at a dose of about 200 mg, about 250 mg, or about 300 mg (per dose) for at least one period of 3 out of 7 days (e.g., 3 consecutive days with dosage followed by 4 consecutive days without dosage).

In another embodiment, the composition is administered twice daily at a dose of about 200 mg, about 250 mg, or about 300 mg (per dose) for at least one period of 4 out of 7 days (e.g., 4 consecutive days with dosage followed by 3 consecutive days without dosage).

In another embodiment, the composition is administered twice daily at a dose of about 200 mg, about 250 mg, or about 300 mg (per dose) for at least one period of 5 out of 7 days (e.g., 5 consecutive days with dosage followed by 2 consecutive days without dosage).

In another embodiment, the composition is administered twice daily at a dose of about 200 mg, about 250 mg, or about 300 mg (per dose) for at least one period of 3 out of 7 days in a cycle of 21 days (e.g., 3 consecutive days or Days 1-3 for up to 3 weeks in a 21 day cycle).

In another embodiment, the composition is administered twice daily at a dose of about 200 mg, about 250 mg, or about 300 mg (per dose) for at least one period of 3 out of 7 days in a cycle of 28 days (e.g., 3 consecutive days or Days 1-3 for up to 4 weeks in a 28 day cycle).

In another embodiment, the composition is administered twice daily at a dose of about 200 mg, about 250 mg, or about 300 mg (per dose) for at least one period of 4 out of 7 days in a cycle of 21 days (e.g., 4 consecutive days or Days 1-4 for up to 3 weeks in a 21 day cycle).

In another embodiment, the composition is administered twice daily at a dose of about 200 mg, about 250 mg, or about 300 mg (per dose) for at least one period of 5 out of 7 days in a cycle of 21 days (e.g., 5 consecutive days or Days 1-5 for up to 3 weeks in a 21 day cycle).

In another embodiment, the composition is administered twice daily at a dose of about 200 mg, about 250 mg, or about 300 mg (per dose), for example, for one period of 3 out of 7 days in a cycle of 21 days (e.g., 3 consecutive days or Days 1-3 in a 21 day cycle).

In another embodiment, the composition is administered twice daily at a dose of about 200 mg, about 250 mg, or about 300 mg (per dose), for example, for at least two periods of 3 out of 7 days in a cycle of 21 days (e.g., 3 consecutive days or Days 1-3 and Days 8-10 for Week 1 and Week 2 of a 21 day cycle).

In another embodiment, the composition is administered twice daily at a dose of about 200 mg, about 250 mg, or about 300 mg (per dose), for example, for at least three periods of 3 out of 7 days in a cycle of 21 days (e.g., 3 consecutive days or Days 1-3, Days 8-10, and Days 15-17 for Week 1, Week 2, and Week 3 of a 21 day cycle).

In another embodiment, the composition is administered twice daily at a dose of about 200 mg, about 250 mg, or about 300 mg (per dose) for at least four periods of 3 out of 7 days in a cycle of 28 days (e.g., 3 consecutive days or Days 1-3, Days 8-10, Days 15-17, and Days 22-24 for Week 1, Week 2, Week 3, and Week 4 in a 28 day cycle).

In another embodiment, the composition is administered twice daily at a dose of about 300 mg (per dose), for example, for at least one period of 7 out of 14 days (e.g., 7 consecutive days or Days 1-7 in a 14 day cycle).

In another embodiment, the composition is administered twice daily at a dose of about 200 mg, about 300 mg, or about 400 mg (per dose), for example, for at least one period of 11 out of 21 days (e.g., 11 consecutive days or Days 1-11 in a 21 day cycle).

In another embodiment, the composition is administered once daily at a dose of about 200 mg, about 300 mg, or about 400 mg (per dose), for example, for at least one period of 10 out of 21 days (e.g., 10 consecutive days or Days 1-10 in a 21 day cycle).

In another embodiment, the composition is administered twice daily at a dose of about 200 mg, about 300 mg, or about 400 mg (per dose), for example, for at least one period of 10 out of 21 days (e.g., 10 consecutive days or Days 1-10 in a 21 day cycle).

In another embodiment, the composition is administered twice daily at a dose of bout 200 mg, about 300 mg, or about 400 mg (per dose), for example, for at least one period of 14 out of 21 days (e.g., 14 consecutive days or Days 1-14 in a 21 day cycle).

In addition, the HDAC inhibitor, retinoid agent, and optional additional anti-cancer agent may be administered according to any of the schedules described above, consecutively for a few weeks, followed by a rest period. For example, the HDAC inhibitor may be administered according to any one of the schedules described above from two to eight weeks, followed by a rest period of one week, or twice daily at a dose of 300 mg for three to five days a week. In another particular embodiment, the HDAC inhibitor can be administered three times daily for two consecutive weeks, followed by one week of rest.

In another aspect of the present invention, treatment procedures comprising administration of an HDAC inhibitor, e.g., SAHA, and a retinoid agent, e.g., Targretin, and optionally another anti-cancer agent, can be performed sequentially in any order, alternating in any order, simultaneously, or any combination thereof. In particular, the administration of an HDAC inhibitor and the administration of the retinoid agent can be performed concurrently, consecutively, or e.g., alternating concurrent and consecutive administration. For example, in one embodiment, the HDAC inhibitor, e.g., SAHA, is administered prior to administering the retinoid agent, e.g., Targretin. In other embodiments, the HDAC inhibitor and the retinoid agent are administered orally.

The HDAC inhibitor, e.g., SAHA, can be pre-administered anywhere from 1 week to four weeks, for one or more months, prior to a concurrent or alternating administration of a retinoid agent, e.g., Targretin, and optionally, another anti-cancer agent.

In another embodiment, the HDAC inhibitor, e.g., SAHA, can be pre-administered at least 1 week prior to a concurrent administration of HDAC inhibitor and retinoid agent, e.g., Targretin, where SAHA is pre-administered or concurrently administered at 400 mg per day. The concurrent administration of SAHA and Targretin can be for six 28-day cycles, or alternatively, SAHA can be administered 400 mg once a day for six 28-day cycles, Targretin can be administered at 150 mg per day for the first 28-day cycle, and at 225 mg per day for the second to sixth 28-day cycle.

In other embodiments, the HDAC inhibitor can be pre-administered or concurrently administered at, inter alia, 100 mg per day, 125 mg per day, 175 mg per day, 200 mg per day, 225 mg per day, 250 mg per day, 275 mg per day, 300 mg per day, 325 mg per day, 350 mg per day, 375 mg per day, 400 mg per day, or more than 400 mg per day. SARA can additionally be administered at any of the dosage amounts once, twice, three, or more than three times daily. Targretin doses can be administered at doses of, inter alia, 50 mg per day, 75 mg per day, 100 mg per day, 125 mg per day, 175 mg per day, 200 mg per day, 225 mg per day, 250 mg per day, 275 mg per day, 300 mg per day, 325 mg per day, 350 mg per day, 375 mg per day, 400 mg per day, 425 mg per day, 450 mg per day, 475 mg per day, 500 mg per day, or more than 500 mg per day.

The HDAC inhibitor, e.g., SAHA, and the retinoid agent, e.g., Targretin, and optionally, another anti-cancer agent can be administered in anywhere from one to twelve 28-day cycles, preferably one to six 28-day cycles, but can also encompass one to eleven 28-day cycles, one to ten 28-day cycles, one to nine 28-day cycles, one to eight 28-day cycles, one to seven 28-day cycles, one to five 28-day cycles, one to four 28-day cycles, one to three 28-day cycles, or one to two 28-day cycles. The SAHA and Targretin cycles can be administered at any dosage combination, and for any combination of 28-day cycles, such as but not limited to, administration of SAHA for six (or more) 28-day cycles and Targretin administration for one 28-day cycle at a first dose (i.e., 150 mg per day), and at a second dose (i.e., 225 mg per day) for the second to sixth (or more) 28-day cycle. Targretin can also be administered at one dose in combination with SAHA (and optionally, another anti-cancer agent) for six (or more) 28-day cycles. Alternatively, Targretin can be administered at a first dose for one 28-day cycle, at a second dose for the second 28-day cycle, and at a third dose for the third to sixth 28-day cycle. Targretin can also be administered at a first dose for one 28-day cycle, at a second dose for the second 28-day cycle, at a third dose for the third 28-day cycle, and at a fourth dose for the fourth to sixth 28-day cycle. Additionally, Targretin can be administered at a first dose for one 28-day cycle, at a second dose for the second 28-day cycle, at a third dose for the third 28-day cycle, at a fourth dose for the fourth 28-day cycle, and at a fifth dose for the fifth and sixth 28-day cycles. Targretin can also be administered at doses that incrementally increase throughout the one or more (preferably six, but up to twelve) 28-day cycles. Such dosing schedules can be determined empirically based on the patient's compliance, progression of disease, age, height, weight, sex, or any other parameter known in the art to affect dosages and/or dosage schedules of anti-cancer agents.

In other embodiments, SAHA and Targretin can be concurrently administered, wherein SAHA is administered 400 mg once a day for six 28-day cycles, Targretin is administered at 150 mg per day for the first 28-day cycle, at 225 mg per day for the second 28-day cycle, and at 300 mg per day for the third to sixth 28-day cycle.

In other embodiments, SAHA and Targretin can be concurrently administered, wherein SAHA is administered 400 mg once a day for six 28-day cycles, Targretin is administered at 150 mg per day for the first 28-day cycle, and at 300 mg per day for the second to sixth 28-day cycle.

SAHA and Targretin, in further embodiments, can be concurrently administered wherein SAHA is administered 400 mg once a day for six 28-day cycles, Targretin is administered at 150 mg per day for the first 28-day cycle, at 300 mg per day for the second 28-day cycle, and at 375 mg per day for the third to sixth 28-day cycle.

In other embodiments, SAHA and Targretin can be concurrently administered, wherein SAHA is administered 400 mg once a day for six 28-day cycles, Targretin is administered at 150 mg per day for the first 28-day cycle, and at 375 mg per day for the second to sixth 28-day cycle.

In other embodiments, SAHA and Targretin can be concurrently administered wherein SAHA is administered 400 mg once a day for six 28-day cycles, Targretin is administered at 150 mg per day for the first 28-day cycle, at 300 mg per day for the second 28-day cycle, and at 450 mg per day for the third to sixth 28-day cycle.

In other embodiments, SAHA and Targretin can be concurrently administered, wherein SAHA is administered 400 mg once a day for six 28-day cycles, Targretin is administered at 150 mg per day for the first 28-day cycle, and at 450 mg per day for the second to sixth 28-day cycle.

In other embodiments, SAHA and Targretin can be concurrently administered, wherein SAHA is administered 400 mg once a day, Targretin is dose escalated from 150 mg per day to 300 mg per day, 375 mg per day or 450 mg per day. In one embodiment, the dose escalation occurs in two, three, four, five or six cycles of 28 days.

In further embodiments, a lipid-lowering agent can be administered during or before the pre-administration period, or a combination thereof. The lipid-lowering agent can be, for example, fenofibrate. Alternatively, thyroxine can be administered at the start of the concurrent administration period. The thyroxine can be, but is not limited to, levothyroxine.

Intravenously or subcutaneously, the patient would receive the HDAC inhibitor in quantities sufficient to deliver between about 3-1500 mg/m² per day, for example, about 3, 30, 60, 90, 180, 300, 600, 900, 1200 or 1500 mg/m² per day. Such quantities may be administered in a number of suitable ways, e.g. large volumes of low concentrations of HDAC inhibitor during one extended period of time or several times a day. The quantities can be administered for one or more consecutive days, intermittent days or a combination thereof per week (7 day period). Alternatively, low volumes of high concentrations of HDAC inhibitor during a short period of time, e.g. once a day for one or more days either consecutively, intermittently or a combination thereof per week (7 day period). For example, a dose of 300 mg/m² per day can be administered for 5 consecutive days for a total of 1500 mg/m² per treatment. In another dosing regimen, the number of consecutive days can also be 5, with treatment lasting for 2 or 3 consecutive weeks for a total of 3000 mg/m² and 4500 mg/m² total treatment.

Typically, an intravenous formulation may be prepared which contains a concentration of HDAC inhibitor of between about 1.0 mg/mL to about 10 mg/mL, e.g. 2.0 mg/mL, 3.0 mg/mL, 4.0 mg/mL, 5.0 mg/mL, 6.0 mg/mL, 7.0 mg/mL, 8.0 mg/mL, 9.0 mg/mL and 10 mg/mL and administered in amounts to achieve the doses described above. In one example, a sufficient volume of intravenous formulation can be administered to a patient in a day such that the total dose for the day is between about 300 and about 1500 mg/m².

In specific aspects, the HDAC inhibitor (e.g., SAHA; Vorinostat) can be administered at a total daily dose of up to 400 mg, and the retinoid agent (e.g., Bexarotene; 3-methyl TTNEB; Targretin) can be administered at a total daily dose at a total daily dose of up to 300 mg/m².

Subcutaneous formulations can be prepared according to procedures well known in the art at a pH in the range between about 5 and about 12, which include suitable buffers and isotonicity agents, as described below. They can be formulated to deliver a daily dose of HDAC inhibitor, retinoid agent, and optional additional anti-cancer agent in one or more daily subcutaneous administrations, e.g., one, two or three times each day.

The HDAC inhibitors, retinoid agents, and optional additional anti-cancer agents can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

The various modes of administration, dosages, and dosing schedules described herein merely set forth specific embodiments and should not be construed as limiting the broad scope of the invention. Any permutations, variations, and combinations of the dosages and dosing schedules are included within the scope of the present invention.

### Administration of Anti-Cancer Agents

The route of administration of SAHA or any one of the other HDAC inhibitors, and Targretin or any other retinoid agent can be independent of the route of administration of the anti-cancer agent. A particular route of administration for SAHA and Targretin is oral administration. Thus, in accordance with this embodiment, SAHA and Targretin are administered orally, and the other anti-cancer agent can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

In addition, the HDAC inhibitor and retinoid agent and optional additional anti-cancer agent may be administered by the same mode of administration, i.e. both agents administered orally, by IV, etc. However, it is also within the scope of the present invention to administer the HDAC inhibitor by one mode of administration, e.g. oral, and to administer the retinoid agent and optional additional anti-cancer agent by another mode of administration, e.g. IV, or any other ones of the administration modes described hereinabove.

Commonly used anti-cancer agents and daily dosages usually administered include but are not restricted to:

| | | | |
|---|---|---|---|
| Antime- | Methotrexate: | 20-40 | mg/m² i.v. |
| tabolites: | Methotrexate: | 4-6 | mg/m² p.o. |
| | Methotrexate: | 12000 | mg/m² |
| | | | high dose therapy |
| | 6-Mercaptopurine: | 100 | mg/m² |
| | 6-Thioguanine: | 1-2 × 80 | mg/m² p.o. |
| | Pentostatin | 4 | mg/m² i.v. |
| | Fludarabin- | 25 | mg/m² i.v. |
| | phosphate: | | |
| | Cladribine: | 0.14 | mg/kg BW i.v. |
| | 5-Fluorouracil | 500-2600 | mg/m² i.v. |
| | Capecitabine: | 1250 | mg/m² p.o. |
| | Cytarabin: | 200 | mg/m² i.v. |
| | Cytarabin: | 3000 | mg/m² i.v. |
| | | | high dose therapy |
| | Gemcitabine: | 800-1250 | mg/m² i.v. |
| | Hydroxyurea: | 800-4000 | mg/m² p.o. |
| Antibiotics: | Actinomycin D | 0.6 | mg/m2 i.v. |
| | Daunorubicin | 45-6.0 | mg/m² i.v. |
| | Doxorubicin | 45-60 | mg/m² i.v. |
| | Epirubicin | 60-80 | mg/m² i.v. |
| | Idarubicin | 10-12 | mg/m² i.v. |
| | Idarubicin | 35-50 | mg/m² p.o. |
| | Mitoxantron | 10-12 | mg/m² i.v. |
| | Bleomycin | 10-15 | mg/m² i.v., i.m., s.c. |
| | Mitomycin C | 10-20 | mg/² i.v. |
| | Irinotecan(CPT-11) | 350 | mg/m² i.v. |
| | Topotecan | 1.5 | mg/m² i.v. |
| Alkylating | Mustargen | 6 | mg/m² i.v. |
| Agents: | Estramustin- | 150-200 | mg/m² i.v. |
| | phosphate | | |
| | Estramustin- | 480-550 | mg/m² p.o. |
| | phosphate | | |
| | Melphalan | 8-10 | mg/m² i.v. |
| | Melphalan | 15 | mg/m² i.v. |
| | Chlorambucil | 3-6 | mg/m² i.v. |
| | Prednimustine | 40-100 | mg/m² p.o. |
| | Cyclophosphamide | 750-1200 | mg/m² i.v. |
| | Cyclophosphamide | 50-100 | mg/m² p.o. |
| | Ifosfamide | 1500-2000 | mg/m² i.v. |
| | Trofosfamide | 25-200 | mg/m² p.o. |
| | Busulfan | 2-6 | mg/m² p.o. |
| | Treosulfan | 5000-8000 | mg/m² i.v. |
| | Treosulfan | 750-1500 | mg/m² p.o. |
| | Thiotepa | 12-16 | mg/m² i.v. |
| | Carmustin (BCNU) | 100 | mg/m² i.v. |
| | Lomustin (CCNU) | 100-130 | mg/m² p.o. |
| | Nimustin (ACNU) | 90-100 | mg/m² i.v. |
| | Dacarbazine (OTIC) | 100-375 | mg/m² i.v. |
| | Procarbazine | 100 | mg/m² p.o. |
| | Cisplatin | 20-120 | mg/m² i.v. |
| | Carboplatin | 300-400 | mg/m² i.v. |
| Antimitotic | Vincristine | 1.5-2 | mg/m² i.v. |
| agents and | Vinblastine | 4-8 | mg/m² i.v. |
| Plant- | Vindesine | 2-3 | mg/m² i.v. |
| derived | Etoposide (VP16) | 100-200 | mg/m² i.v. |
| agents: | Etoposide (VP16) | 100 | mg p.o. |
| | Teniposide (VM26) | 20-30 | mg/m² i.v. |
| | Paclitaxel (Taxol) | 175-250 | mg/m² i.v. |
| | Docetaxel (Taxotere) | 100-150 | mg/m² i.v. |
| Hormones, | Interferon-α | 2-10 × 10⁶ | IU/m² |
| Cytokines | Prednisone | 40-100 | mg/m² p.o. |
| and | Dexamethasone | 8-24 | mg p.o. |
| Vitamins: | G-CSF | 5-20 | μg/kg BW s.c. |
| | all-trans Retinoic | 45 | mg/m² |
| | Acid | | |
| | Interleukin-2 | 18 × 10⁶ | IU/m² |
| | GM-CSF | 250 | mg/m² |
| | Erythropoietin | 150 | IU/kg tiw |

The dosage regimens utilizing the anti-cancer agents described herein (or any pharmaceutically acceptable salts or hydrates of such agents, or any free acids, free bases, or other free forms of such agents) can follow the exemplary dosages herein, including those provided for HDAC inhibitors. The dosage can be selected in accordance with a variety of factors including type, species, age, weight, sex and the type of disease being treated; the severity (i.e., stage) of the disease to be treated; the route of administration; the renal and hepatic function of the patient; and the particular compound or salt thereof employed. A dosage regiment can be used, for example, to treat, for example, to prevent, inhibit (fully or partially), or arrest the progress of the disease.

In particular embodiments, a retinoid agent is administered in a dose from about 0.05 mg/kg to about 7.5 mg/kg or about 1.5 mg/kg to about 7.5 mg/kg. As a specific example, liposomal ATRA may be administered in a dose from about 15 mg/m² to 75 mg/m².

### Combination Administration

In accordance with the invention, HDAC inhibitors, retinoid agents, and additional anti-cancer agents can be used in the treatment of a wide variety of cancers, including but not limited to solid tumors (e.g., tumors of the head and neck, lung, breast, colon, prostate, bladder, rectum, brain, gastric tissue, bone, ovary, thyroid, or endometrium), hematological malignancies (e.g., leukemias, lymphomas, myelomas), carcinomas (e.g. bladder carcinoma, renal carcinoma, breast carcinoma, colorectal carcinoma), neuroblastoma, or melanoma. Non-limiting examples of these cancers include diffuse large B-cell lymphoma (DLBCL), T-cell lymphomas or leukemias, e.g., cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), as well as acute lymphocytic leukemia, acute nonlymphocytic leukemia, acute myeloid leukemia, chronic lymphocytic leukemia, chronic myelogenous leukemia, Hodgkin's disease, non-Hodgkin's lymphoma, myeloma, multiple myeloma, mesothelioma, childhood solid tumors, neuroblastoma, retinoblastoma, glioma, Wilms' tumor, bone cancer and soft-tissue sarcomas, common solid tumors of adults such as head and neck cancers (e.g., oral, laryngeal and esophageal), genitourinary cancers (e.g., prostate, bladder, renal, uterine, ovarian, testicular, rectal and colon), lung cancer (e.g., small cell carcinoma and non-small cell lung carcinoma, including squamous cell carcinoma and adenocarcinoma), breast cancer, pancreatic cancer, melanoma and other skin cancers, stomach cancer, brain cancer, liver cancer, adrenal cancer, kidney cancer, thyroid cancer, basal cell carcinoma, squamous cell carcinoma of both ulcerating and papillary type, metastatic skin carcinoma, medullary carcinoma, osteo sarcoma, Ewing's sarcoma, veticulum cell sarcoma, and Kaposi's sarcoma. Also included are pediatric forms of any of the cancers described herein.

Cutaneous T-cell lymphomas and peripheral T-cell lymphomas are forms of non-Hodgkin's lymphoma. Cutaneous T-cell lymphomas are a group of lymphoproliferative disorders characterized by localization of malignant T lymphocytes to the skin at presentation. CTCL frequently involves the skin, bloodstream, regional lymph nodes and spleen. Mycosis flugoides (MF), the most common and indolent form of CTCL, is characterized by patches, plaques or tumors containing epidermotropic CD4⁺CD45RO⁺ helper/memory T cells. MF may evolve into a leukemic variant, Sézary syndrome (SS), or transform to large cell lymphoma. The condition causes severe skin itching, pain and edema.

Currently, CTCL is treated topically with steroids, photochemotherapy and chemotherapy, as well as radiotherapy. Peripheral T-cell lymphomas originate from mature or peripheral (not central or thymic) T-cell lymphocytes as a clonal proliferation from a single T-cell and are usually either predominantly nodal or extranodal tumors. They have T-cell lymphocyte cell-surface markers and clonal arrangements of the T-cell receptor genes. Approximately 16,000 to 20,000 people in the U.S. are affected by either CTCL or PTCL. These diseases are highly symptomatic. Patches, plaques and tumors are the clinical names of the different presentations. Patches are usually flat, possibly scaly and look like a “rash.” Mycosis fungoides patches are often mistaken for eczema, psoriasis or non-specific dermatitis until a proper diagnosis of mycosis fungoides is made. Plaques are thicker, raised lesions. Tumors are raised “bumps” which may or may not ulcerate. A common characteristic is itching or pruritus, although many patients do not experience itching. It is possible to have one or all three of these phases. For most patients, existing treatments are palliative but not curative.

According to the National Cancer Institute, head and neck cancers account for three percent of all cancers in the U.S. Most head and neck cancers originate in the squamous cells lining the structures found in the head and neck, and are often referred to as squamous cell carcinomas of the head and neck (SCCHN). Some head and neck cancers originate in other types of cells, such as glandular cells. Head and neck cancers that originate in glandular cells are called adenocarcinomas. Head and neck cancers are further defined by the area in which they begin, such as the oral cavity, nasal cavity, larynx, pharynx, salivary glands, and lymph nodes of the upper part of the neck. It is estimated that 38,000 people in the U.S. developed head and neck cancer 2002. Approximately 60% of patients present with locally advanced disease. Only 30% of these patients achieve long-term remission after treatment with surgery and/or radiation. For patients with recurrent and/or metastatic disease, the median survival is approximately six months.

Alkylating agents suitable for use in the present invention include but are not limited to bischloroethylamines (nitrogen mustards, e.g., Chlorambucil, Cyclophosphamide, Ifosfamide, Mechlorethamine, Melphalan, uracil mustard), aziridines (e.g., Thiotepa), alkyl alkone sulfonates (e.g., Busulfan), nitrosoureas (e.g., Carmustine, Lomustine, Streptozocin), nonclassic alkylating agents (e.g., Altretamine, Dacarbazine, and Procarbazine), platinum compounds (e.g., Carboplastin and Cisplatin).

Antibiotic agents suitable for use in the present invention are anthracyclines (e.g., Doxorubicin, Daunorubicin, Epirubicin, Idarubicin, and Anthracenedione), Mitomycin C, Bleomycin, Dactinomycin, Plicatomycin.

Antimetabolic agents suitable for use in the present invention include but are not limited to Floxuridine, Fluorouracil, Methotrexate, Leucovorin, Hydroxyurea, Thioguanine, Mercaptopurine, Cytarabine, Pentostatin, Fludarabine Phosphate, Cladribine, Asparaginase, and Gemcitabine. In a particular embodiment, the antimetabolic agent in Gemcitabine.

Hormonal agents suitable for use in the present invention, include but are not limited to, an estrogen, a progestogen, an antiesterogen, an androgen, an antiandrogen, an LHRH analogue, an aromatase inhibitor, Diethylstibestrol, Tamoxifen, Toremifene, Fluoxymesterol, Raloxifene, Bicalutamide, Nilutamide, Flutamide, Aminoglutethimide, Tetrazole, Ketoconazole, Goserelin Acetate, Leuprolide, Megestrol Acetate, and Mifepristone.

Plant-derived agents suitable for use in the present invention include, but are not limited to Vincristine, Vinblastine, Vindesine, Vinzolidine, Vinorelbine, Etoposide Teniposide, Paclitaxel, and Docetaxel.

Biologic agents suitable for use in the present invention include, but are not limited to immuno-modulating proteins, monoclonal antibodies against tumor antigens, tumor suppressor genes, and cancer vaccines. For example, the immuno-modulating protein can be interleukin 2 (IL-2), interleukin 4 (IL-4), interleukin 12 (IL-12), interferon E1 (IFN E1), interferon D (IFN-D), interferon alpha (IFN-α), interferon beta (IFN-β), interferon gamma (IFN-γ), erythropoietin (EPO), granulocyte-CSF (G-CSF), macrophage-CSF (M-CSF), granulocyte-macrophage-CSF (GM-CSF), bacillus Calmette-Guerin, Levamisole, or Octreotide. Furthermore, the tumor suppressor gene can be DPC-4, NF-1, NF-2, RB, p53, WT1, BRCA, or BRCA2.

In various aspects of the invention, the treatment procedures are performed sequentially in any order, simultaneously, or a combination thereof. For example, the first treatment procedure, e.g., administration of an HDAC inhibitor, can take place prior to the second treatment procedure, e.g., the retinoid agent, after the second treatment with the retinoid agent, at the same time as the second treatment with the retinoid agent, or a combination thereof. The treatment procedures can also include an optional third treatment procedure, e.g., administration of another anti-cancer agent, which can take place prior to the second treatment procedure, e.g., the retinoid agent, after the second treatment with the retinoid agent, at the same time as the second treatment with the retinoid agent, prior to the first treatment procedure, e.g., the HDAC inhibitor, after the first treatment with the HDAC inhibitor, at the same time as the first treatment, at the same time as the first and second treatment, or combinations thereof.

In one aspect of the invention, a total treatment period can be decided for the HDAC inhibitor. The retinoid agent and optional additional anti-cancer agent can be administered prior to onset of treatment with the HDAC inhibitor or following treatment with the HDAC inhibitor. In addition, the retinoid agent and optional additional anti-cancer agent can be administered during the period of HDAC inhibitor administration but does not need to occur over the entire HDAC inhibitor treatment period. Similarly, the HDAC inhibitor can be administered prior to onset of treatment with the retinoid agent and optional additional anti-cancer agent or following treatment with the retinoid agent and optional additional anti-cancer agent. In addition, the HDAC inhibitor can be administered during the period of retinoid agent and optional additional anti-cancer agent administration but does not need to occur over the entire retinoid agent and optional additional anti-cancer agent treatment period. Alternatively, the treatment regimen includes pre-treatment with one agent, either the HDAC inhibitor or the retinoid agent and/or optional additional anti-cancer agent, followed by the addition of the other agent(s) for the duration of the treatment period.

In a particular embodiment, the combination of the HDAC inhibitor and retinoid agent and optional additional anti-cancer agent is additive, i.e., the combination treatment regimen produces a result that is the additive effect of each constituent when it is administered alone. In accordance with this embodiment, the amount of HDAC inhibitor and the amount of the retinoid agent and optional additional anti-cancer together constitute an effective amount to treat cancer.

In another embodiment, the combination of the HDAC inhibitor and retinoid agent and optional additional anti-cancer agent is considered therapeutically synergistic when the combination treatment regimen produces a significantly better anticancer result (e.g., cell growth arrest, apoptosis, induction of differentiation, cell death) than the additive effects of each constituent when it is administered alone at a therapeutic dose. Standard statistical analysis can be employed to determine when the results are significantly better. For example, a Mann-Whitney Test or some other generally accepted statistical analysis can be employed.

In one particular embodiment of the present invention, the HDAC inhibitor can be administered in combination with an additional HDAC inhibitor. In another particular embodiment of the present invention, the HDAC inhibitor can be administered in combination with a retinoid agent and optionally, an alkylating agent. In another particular embodiment of the present invention, the HDAC inhibitor and retinoid agent can be administered in combination with an antibiotic agent. In another particular embodiment of the present invention, the HDAC inhibitor and retinoid agent can be administered in combination with an antimetabolic agent. In another particular embodiment of the present invention, the HDAC inhibitor and retinoid agent can be administered in combination with a hormonal agent. In another particular embodiment of the present invention, the HDAC inhibitor and retinoid agent can be administered in combination with a plant-derived agent.

In another particular embodiment of the present invention, the HDAC inhibitor and retinoid agent can be administered in combination with an anti-angiogenic agent. In another particular embodiment of the present invention, the HDAC inhibitor and retinoid agent can be administered in combination with a differentiation inducing agent.

In another particular embodiment of the present invention, the HDAC inhibitor and retinoid agent can be administered in combination with a cell growth arrest inducing agent. In another particular embodiment of the present invention, the HDAC inhibitor and retinoid agent can be administered in combination with an apoptosis inducing agent. In another particular embodiment of the present invention, the HDAC inhibitor and retinoid agent can be administered in combination with a cytotoxic agent. In another particular embodiment of the present invention, the HDAC inhibitor and retinoid agent can be administered in combination with another retinoid agent. In another particular embodiment of the present invention, the HDAC inhibitor and retinoid agent can be administered in combination with a biologic agent. In another particular embodiment of the present invention, the HDAC inhibitor and retinoid agent can be administered in combination with any combination of an additional HDAC inhibitor, an alkylating agent, an antibiotic agent, an antimetabolic agent, a hormonal agent, a plant-derived agent, an anti-angiogenic agent, a differentiation inducing agent, a cell growth arrest inducing agent, an apoptosis inducing agent, a cytotoxic agent, an additional retinoid agent or a biologic agent.

The combination therapy can act through the induction of cancer cell differentiation, cell growth arrest, and/or apoptosis. The combination of therapy is particularly advantageous, since the dosage of each agent in a combination therapy can be reduced as compared to monotherapy with the agent, while still achieving an overall anti-tumor effect.

### Pharmaceutical Compositions

As described above, the compositions comprising the HDAC inhibitor, retinoid agent, and/or the additional anti-cancer agent can be formulated in any dosage form suitable for oral, parenteral, intraperitoneal, intravenous, intraarterial, transdermal, sublingual, intramuscular, rectal, transbuccal, intranasal, liposomal, via inhalation, vaginal, or intraocular administration, for administration via local delivery by catheter or stent, or for subcutaneous, intraadiposal, intraarticular, intrathecal administration, or for administration in a slow release dosage form.

The HDAC inhibitor, retinoid agent, and optional additional anti-cancer agent can be formulated in the same formulation for simultaneous administration, or they can be in two separate dosage forms, which may be administered simultaneously or sequentially as described above.

The invention also encompasses pharmaceutical compositions comprising pharmaceutically acceptable salts of the HDAC inhibitors, retinoid agents, and/or optional additional anti-cancer agents.

Suitable pharmaceutically acceptable salts of the compounds described herein and suitable for use in the method of the invention, are conventional non-toxic salts and can include a salt with a base or an acid addition salt such as a salt with an inorganic base, for example, an alkali metal salt (e.g., lithiwn salt, sodium salt, potassium salt, etc.), an alkaline earth metal salt (e.g., calcium salt, magnesium salt, etc.), an ammonium salt; a salt with an organic base, for example, an organic amine salt (e.g., triethylamine salt, pyridine salt, picoline salt, ethanolamine salt, triethanolamine salt, dicyclohexylamine salt, N,N′-dibenzylethylenediamine salt, etc.) etc.; an inorganic acid addition salt (e.g., hydrochloride, hydrobromide, sulfate, phosphate, etc.); an organic carboxylic or sulfonic acid addition salt (e.g., formate, acetate, trifluoroacetate, maleate, tartrate, methanesulfonate, benzenesulfonate, p-toluenesulfonate, etc.); a salt with a basic or acidic amino acid (e.g., arginine, aspartic acid, glutamic acid, etc.) and the like.

The invention also encompasses pharmaceutical compositions comprising hydrates of the HDAC inhibitors, retinoid agents, and/or optional additional anti-cancer agents.

In addition, this invention also encompasses pharmaceutical compositions comprising any solid or liquid physical form of SAHA or any of the other HDAC inhibitors in combination with any solid or liquid physical form of Targretin or any other retinoid agent (and optionally, another anti-cancer agent). For example, The HDAC inhibitors and retinoid agents (and optionally, another anti-cancer agent) can be in a crystalline form, in amorphous form, and have any particle size. The HDAC inhibitor and retinoid agent particles (and optionally, another anti-cancer agent) may be micronized, or may be agglomerated, particulate granules, powders, oils, oily suspensions or any other form of solid or liquid physical form.

For oral administration, the pharmaceutical compositions can be liquid or solid. Suitable solid oral formulations include tablets, capsules, pills, granules, pellets, and the like. Suitable liquid oral formulations include solutions, suspensions, dispersions, emulsions, oils, and the like.

Any inert excipient that is commonly used as a carrier or diluent may be used in the formulations of the present invention, such as for example, a gum, a starch, a sugar, a cellulosic material, an acrylate, or mixtures thereof. The compositions may further comprise a disintegrating agent and a lubricant, and in addition may comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof. Furthermore, the compositions of the present invention may be in the form of controlled release or immediate release formulations.

The HDAC inhibitors, retinoid agents, and optional additional anti-cancer agents can be administered as active ingredients in admixture with suitable pharmaceutical diluents, excipients or carriers (collectively referred to herein as “carrier” materials or “pharmaceutically acceptable carriers”) suitably selected with respect to the intended form of administration. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions, or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide.

Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCI, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), a glidant (e.g., colloidal silicon dioxide), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The preparation of pharmaceutical compositions that contain an active component is well understood in the art, for example, by mixing, granulating, or tablet-forming processes. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic, or oily solutions and the like as detailed above.

The amount of the compound(s) administered to the patient is less than an amount that would cause unmanageable toxicity in the patient. In the certain embodiments, the amount of the compound(s) that is administered to the patient is less than the amount that causes a concentration of the compound in the patient's plasma to equal or exceed the toxic level of the compound. In particular embodiments, the concentration of the compound(s) in the patient's plasma is maintained at about 10 nM. In another embodiment, the concentration of the compound(s) in the patient's plasma is maintained at about 25 nM. In another embodiment, the concentration of the compound(s) in the patient's plasma is maintained at about 50 nM. In another embodiment, the concentration of the compound(s) in the patient's plasma is maintained at about 100 nM. In another embodiment, the concentration of the compound(s) in the patient's plasma is maintained at about 500 nM. In another embodiment, the concentration of the compound(s) in the patient's plasma is maintained at about 1,000 nM. In another embodiment, the concentration of the compound(s) in the patient's plasma is maintained at about 2,500 nM. In another embodiment, the concentration of the compound(s) in the patient's plasma is maintained at about 5,000 nM. The optimal amount of the compound(s) that should be administered to the patient in the practice of the present invention will depend on the particular compound(s) used and the type of cancer being treated.

The percentage of the active ingredients and various excipients in the formulations may vary. For example, the composition may comprise between 20 and 90%, or specifically between 50-70% by weight of active agent(s).

For IV administration, Glucuronic acid, L-lactic acid, acetic acid, citric acid or any pharmaceutically acceptable acid/conjugate base with reasonable buffering capacity in the pH range acceptable for intravenous administration can be used as buffers. Sodium chloride solution wherein the pH has been adjusted to the desired range with either acid or base, for example, hydrochloric acid or sodium hydroxide, can also be employed. Typically, a pH range for the intravenous formulation can be in the range of from about 5 to about 12. A particular pH range for intravenous formulation comprising an HDAC inhibitor wherein the HDAC inhibitor has a hydroxamic acid moiety, can be about 9 to about 12.

Subcutaneous formulations can be prepared according to procedures well known in the art at a pH in the range between about 5 and about 12, which include suitable buffers and isotonicity agents. They can be formulated to deliver a daily dose of the active agent in one or more daily subcutaneous administrations. The choice of appropriate buffer and pH of a formulation, depending on solubility of the HDAC inhibitor and retinoid agent (and optionally, another anti-cancer agent) to be administered, is readily made by a person having ordinary skill in the art. Sodium chloride solution wherein the pH has been adjusted to the desired range with either acid or base, for example, hydrochloric acid or sodium hydroxide, can also be employed in the subcutaneous formulation. Typically, a pH range for the subcutaneous formulation can be in the range of from about 5 to about 12. A particular pH range for subcutaneous formulation of an HDAC inhibitor having a hydroxamic acid moiety can be about 9 to about 12.

The compositions of the present invention can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

The present invention also provides in-vitro methods for selectively inducing terminal differentiation, cell growth arrest and/or apoptosis of neoplastic cells, thereby inhibiting proliferation of such cells, by contacting the cells with a first amount of suberoylanilide hydroxamic acid (SAHA) or a pharmaceutically acceptable salt or hydrate thereof, and a second amount of a retinoid agent, and optionally a third amount of another anti-cancer agent, wherein the first and second (and optionally third) amounts together comprise an amount effective to induce terminal differentiation, cell growth arrest of apoptosis of the cells.

Although the methods of the present invention can be practiced in vitro, it is contemplated that a particular embodiment for the methods of selectively inducing terminal differentiation, cell growth arrest and/or apoptosis of neoplastic cells will comprise contacting the cells in vivo, i.e., by administering the compounds to a subject harboring neoplastic cells or tumor cells in need of treatment.

As such, the present invention also provides methods for selectively inducing terminal differentiation, cell growth arrest and/or apoptosis of neoplastic cells, thereby inhibiting proliferation of such cells in a subject by administering to the subject a first amount of suberoylanilide hydroxamic acid (SAHA) or a pharmaceutically acceptable salt or hydrate thereof, in a first treatment procedure, and a second amount of a retinoid agent in a second treatment procedure, and optionally, a third amount of an anti-cancer agent in a third treatment procedure, wherein the first and second (and optionally third) amounts together comprise an amount effective to induce terminal differentiation, cell growth arrest of apoptosis of the cells.

The invention is illustrated in the examples that follow. This section is set forth to aid in an understanding of the invention but is not intended to, and should not be construed to limit in any way the invention as set forth in the claims which follow thereafter.

### EXAMPLES

The examples are presented in order to more fully illustrate the various embodiments of the invention. These examples should in no way be construed as limiting the scope of the invention recited in the appended claims.

### Example 1

### Synthesis of SAHA

SAHA can be synthesized according to the method outlined below, or according to the method set forth in U.S. Pat. No. 5,369,108, the contents of which are incorporated by reference in their entirety, or according to any other method.

### Synthesis of SAHA

### Step 1—Synthesis of Suberanilic acid

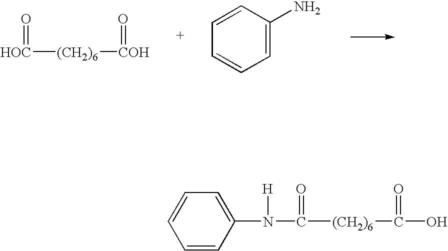

In a 22 L flask was placed 3,500 g (20.09 moles) of suberic acid, and the acid melted with heat. The temperature was raised to 175° C., and then 2,040 g (21.92 moles) of aniline was added. The temperature was raised to 190° C. and held at that temperature for 20 minutes. The melt was poured into a Nalgene tank that contained 4,017 g of potassium hydroxide dissolved in 50 L of water. The mixture was stirred for 20 minutes following the addition of the melt. The reaction was repeated at the same scale, and the second melt was poured into the same solution of potassium hydroxide. After the mixture was thoroughly stirred, the stirrer was turned off, and the mixture was allowed to settle.

The mixture was then filtered through a pad of Celite (4,200 g). The product was filtered to remove the neutral by-product from attack by aniline on both ends of suberic acid. The filtrate contained the salt of the product, and also the salt of unreacted suberic acid. The mixture was allowed to settle because the filtration was very slow, taking several days. The filtrate was acidified using 5 L of concentrated hydrochloric acid; the mixture was stirred for one hour, and then allowed to settle overnight. The product was collected by filtration, and washed on the funnel with deionized water (4×5 L). The wet filter cake was placed in a 72 L flask with 44 L of deionized water, the mixture heated to 50° C., and the solid isolated by a hot filtration (the desired product was contaminated with suberic acid which is has a much greater solubility in hot water. Several hot triturations were done to remove suberic acid. The product was checked by NMR [D₆DMSO] to monitor the removal of suberic acid). The hot trituration was repeated with 44 L of water at 50° C. The product was again isolated by filtration, and rinsed with 4 L of hot water. It was dried over the weekend in a vacuum oven at 65° C. using a Nash pump as the vacuum source (the Nash pump is a liquid ring pump (water) and pulls a vacuum of about 29 inch of mercury. An intermittent argon purge was used to help carry off water); 4,182.8 g of suberanilic acid was obtained.

The product still contained a small amount of suberic acid; therefore the hot trituration was done portionwise at 65° C., using about 300 g of product at a time. Each portion was filtered, and rinsed thoroughly with additional hot water (a total of about 6 L). This was repeated to purify the entire batch. This completely removed suberic acid from the product. The solid product was combined in a flask and stirred with 6 L of methanol/water (1:2), and then isolated by filtration and air dried on the filter over the week end. It was placed in trays and dried in a vacuum oven at 65° C. for 45 hours using the Nash pump and an argon bleed. The final product has a weight of 3,278.4 g (32.7% yield).

### Step 2—Synthesis of Methyl Suberanilate

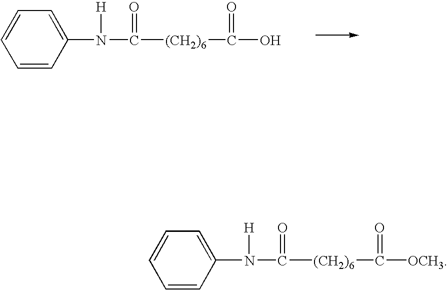

To a 50 L flask fitted with a mechanical stirrer, and condenser was placed 3,229 g of suberanilic acid from the previous step, 20 L of methanol, and 398.7 g of Dowex 50WX2-400 resin. The mixture was heated to reflux and held at reflux for 18 hours. The mixture was filtered to remove the resin beads, and the filtrate was taken to a residue on a rotary evaporator.

The residue from the rotary evaporator was transferred into a 50 L flask fitted with a condenser and mechanical stirrer. To the flask was added 6 L of methanol, and the mixture heated to give a solution. Then 2 L of deionized water was added, and the heat turned off. The stirred mixture was allowed to cool, and then the flask was placed in an ice bath, and the mixture cooled. The solid product was isolated by filtration, and the filter cake was rinsed with 4 L of cold methanol/water (1:1). The product was dried at 45° C. in a vacuum oven using a Nash pump for a total of 64 hours to give 2,850.2 g (84% yield) of methyl suberanilate.

### Step 3—Synthesis of Crude SAHA

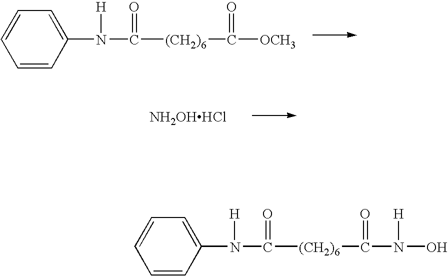

To a 50 L flask with a mechanical stirrer, thermocouple, and inlet for inert atmosphere was added 1,451.9 g of hydroxylamine hydrochloride, 19 L of anhydrous methanol, and a 3.93 L of a 30% sodium methoxide solution in methanol. The flask was then charged with 2,748.0 g of methyl suberanilate, followed by 1.9 L of a 30% sodium methoxide solution in methanol. The mixture was allowed to stir for 16 hr and 10 minutes. Approximately one half of the reaction mixture was transferred from the reaction flask (flask 1) to a 50 L flask (flask 2) fitted with a mechanical stirrer. Then 27 L of deionized water was added to flask 1 and the mixture was stirrer for 10 minutes. The pH was taken using a pH meter; the pH was 11.56. The pH of the mixture was adjusted to 12.02 by the addition of 100 ml of the 30% sodium methoxide solution in methanol; this gave a clear solution (the reaction mixture at this time contained a small amount of solid. The pH was adjusted to give a clear solution from which the precipitation the product would be precipitated). The reaction mixture in flask 2 was diluted in the same manner; 27 L of deionized water was added, and the pH adjusted by the addition of 100 ml of a 30% sodium methoxide solution to the mixture, to give a pH of 12.01 (clear solution).

The reaction mixture in each flask was acidified by the addition of glacial acetic acid to precipitate the product. Flask 1 had a final pH of 8.98, and Flask 2 had a final pH of 8.70. The product from both flasks was isolated by filtration using a Buchner funnel and filter cloth. The filter cake was washed with 15 L of deionized water, and the funnel was covered and the product was partially dried on the funnel under vacuum for 15.5 hr. The product was removed and placed into five glass trays. The trays were placed in a vacuum oven and the product was dried to constant weight. The first drying period was for 22 hours at 60° C. using a Nash pump as the vacuum source with an argon bleed. The trays were removed from the vacuum oven and weighed. The trays were returned to the oven and the product dried for an additional 4 hr and 10 minutes using an oil pump as the vacuum source and with no argon bleed. The material was packaged in double 4-mill polyethylene bags, and placed in a plastic outer container. The final weight after sampling was 2633.4 g (95.6%).

### Step 4—Recrystallization of Crude SAHA

The crude SAHA was recrystallized from methanol/water. A 50 L flask with a mechanical stirrer, thermocouple, condenser, and inlet for inert atmosphere was charged with the crude SAHA to be crystallized (2,525.7 g), followed by 2,625 ml of deionized water and 15,755 ml of methanol. The material was heated to reflux to give a solution. Then 5,250 ml of deionized water was added to the reaction mixture. The heat was turned off, and the mixture was allowed to cool. When the mixture had cooled sufficiently so that the flask could be safely handled (28° C.), the flask was removed from the heating mantle, and placed in a tub for use as a cooling bath. Ice/water was added to the tub to cool the mixture to −5° C. The mixture was held below that temperature for 2 hours. The product was isolated by filtration, and the filter cake washed with 1.5 L of cold methanol/water (2:1). The funnel was covered, and the product was partially dried under vacuum for 1.75 hr. The product was removed from the funnel and placed in 6 glass trays. The trays were placed in a vacuum oven, and the product was dried for 64.75 hr at 60° C. using a Nash pump as the vacuum source, and using an argon bleed. The trays were removed for weighing, and then returned to the oven and dried for an additional 4 hours at 60° C. to give a constant weight. The vacuum source for the second drying period was an oil pump, and no argon bleed was used. The material was packaged in double 4-mill polyethylene bags, and placed in a plastic outer container. The final weight after sampling was 2,540.9 g (92.5%).

In other experiments, crude SAHA was crystallized using the following conditions:

TABLE 2
SAHA Crystallization Conditions

| Solvent | Water | Agitation | Time (hr) |
|---|---|---|---|
| Methanol | — | Off | 2 |
| Methanol | — | On | 72 |
| Ethanol | — | On | 72 |
| Isopropanol | — | Off | 72 |
| Ethanol | 15% | On | 2 |
| Methanol | 15% | Off | 72 |
| Ethanol | 15% | Off | 72 |
| Ethanol | 15% | On | 72 |
| Methanol | 15% | On | 72 |

All these reaction conditions produced SAHA Polymorph I.

### Example 2

### Generation of Wet-Milled Small Particles in 1:1 Ethanol/Water

The SAHA Polymorph I crystals were suspended in 1:1 (by volume) EtOH/water solvent mixture at a slurry concentration ranging from 50 mg/gram to 150 mg/gram (crystal/solvent mixture). The slurry was wet milled with IKA-Works Rotor-Stator high shear homogenizer model T50 with superfine blades at 20-30 m/s, until the mean particle size of SAHA was less than 50 μm and 95% less than 100 μm, while maintaining the temperature at room temperature. The wet-milled slurry was filtered and washed with the 1:1 EtOH/water solvent mixture at room temperature. The wet cake was then dried at 40° C. The final mean particle size of the wet-milled material was less than 50 μm as measured by the Microtrac method below.

Particle size was analyzed using an SRA-150 laser diffraction particle size analyzer, manufactured by Microtrac Inc. The analyzer was equipped with an ASVR (Automatic Small Volume Recirculator). Lecithin at 0.25 wt % in ISOPAR G was used as the dispersing fluid. Three runs were recorded for each sample and an average distribution was calculated. Particle size distribution (PSD) was analyzed as a volume distribution. The mean particle size and 95%<values based on volume were reported.

### Example 2A

### Large Scale Generation of Wet-Milled Small Particles in 1:1 Ethanol/Water

SAHA Polymorph I crystals (56.4 kg) were charged to 610 kg (10.8 kg solvent per kg SAHA) of a 50% vol/vol solution of 200 proof punctilious ethanol and water (50/50 EtOH/Water) at 20-25° C. The slurry (˜700 L) was recirculated through an IKA Works wet-mill set with super-fine generators until reaching a steady-state particle size distribution. The conditions were: DR3-6, 23 m/s rotor tip speed, 30-35 Lpm, 3 gen, ˜96 turnovers (a turnover is one batch volume passed through one gen), ˜12 hrs.

$$Approx . Mill Time ( hr ) = \frac{96 \times Batch Volume ( L )}{\begin{matrix}Natural Draft of Mill ( Lpm ) \times \\ \# of Generators \times 60\end{matrix}}$$

The wet cake was filtered, washed 2× with water (total 6 kg/kg, ˜340 kg) and vacuum dried at 40-45° C. The dry cake was then sieved (595 μm screen) and packed as Fine API.

### Example 3

### Growth of Large Crystals of Mean Particle Size 150 μm in 1:1 Ethanol/Water

Twenty-five grams of SAHA Polymorph I crystals and 388 grams of 1:1 Ethanol/water solvent mixture were charged into a 500 ml jacketed resin kettle with a glass agitator. The slurry was wet milled to a particle size less than 50 μm at room temperature following the steps of Example 2. The wet-milled slurry was heated to 65° C. to dissolve ˜85% of the solid. The heated slurry was aged at 65° C. for 1-3 hours to establish a ˜15% seed bed. The slurry was mixed in the resin kettle under 20 psig pressure, and at an agitator speed range of 400-700 rpm.

The batch was then cooled slowly to 5° C.: 65 to 55° C. in 10 hours, 55 to 45° C. in 10 hours, 45 to 5° C. in 8 hours. The cooled batch was aged at 5° C. for one hour to reach a target supernatant concentration of less than 5 mg/g, in particular, 3 mg/g. The batch slurry was filtered and washed with 1:1 EtOH/water solvent mixture at 5° C. The wet cake was dried at 40° C. under vacuum. The dry cake had a final particle size of 150 μm with 95% particle size<300 μm according to the Microtrac method.

### Example 4

### Growth of Large Crystals with Mean Particle Size of 140 μm in 1:1 Ethanol/Water

SAHA Polymorph I crystals at 7.5 grams and 70.7 grams of 1:1 EtOH/water solvent mixture were charged into a seed preparation vessel (500-ml jacketed resin kettle). The seed slurry was wet milled to a particle size less than 50 μm at room temperature following the steps of Example 2 above. The seed slurry was heated to 63-67° C. and aged over 30 minutes to 2 hours.

In a separate crystallizer (1-liter jacketed resin kettle), 17.5 grams of SAHA Polymorph I crystals and 317.3 grams of 1:1 EtOH/water solvent mixture were charged. The crystallizer was heated to 67-70° C. to dissolve all solid SAHA crystals first, and then was cooled to 60-65° C. to keep a slightly supersaturated solution.

The seed slurry from the seed preparation vessel was transferred to the crystallizer. The slurry was mixed in the resin kettle under 20 psig pressure, and at an agitator speed range similar to that in Example 3. The batch slurry was cooled slowly to 5° C. according to the cooling profile in Example 3. The batch slurry was filtered and washed with 1:1 EtOH/water solvent mixture at 5° C. The wet cake was dried at 40° C. under vacuum. The dry cake had a final particle size of about 140 μm with 95% particle size<280 μm.

### Example 4A

### Large Scale Growth of Large Crystals in 1:1 Ethanol/Water

The Fine API dry cake (21.9 kg) from Example 2A (30% of total) and 201 kg of 50/50 EtOH/Water solution (2.75 kg solvent/kg total SARA) was charged to Vessel #1—the Seed Preparation Tank. SAHA Polymorph I crystals (51.1 kg; 70% of total) and 932 kg 50/50 EtOH/Water (12.77 kg solvent/kg total SAHA) was charged to Vessel #2—the Crystallizer. The Crystallizer was pressurized to 20-25 psig and the contents heated to 67-70° C. while maintaining the pressure to fully dissolve the crystalline SAHA. The contents were then cooled to 61-63° C. to supersaturate the solution. During the aging process in the Crystallizer, the Seed Prep Tank was pressurized to 20-25 psig, the seed slurry was heated to 64° C. (range: 62-66° C.), aged for 30 minutes while maintaining the pressure to dissolve ˜½ of the seed solids, and then cooled to 61-63° C.

The hot seed slurry was rapidly transferred from the Seed Prep Tank to the Crystallizer (no flush) while maintaining both vessel temperatures. The nitrogen pressure in the Crystallizer was re-established to 20-25 psig and the batch was aged for 2 hours at 61-63° C. The batch was cooled to 5° C. in three linear steps over 26 hours: (1) from 62° C. to 55° C. over 10 hours; (2) from 55° C. to 45° C. over 6 hours; and (3) from 45° C. to 5° C. over 10 hours. The batch was aged for 1 hr and then the wet cake was filtered and washed 2× with water (total 6 kg/kg, ˜440 kg), and vacuum dried at 40-45° C. The dry cake from this recrystallization process is packed-out as the Coarse API. Coarse API and Fine API were blended at a 70/30 ratio.

### Example 5

### Generation of Wet-milled Small Particles Batch 288

SAHA Polymorph I crystals were suspended in ethanolic aqueous solution (100% ethanol to 50% ethanol in water by volume) at a slurry concentration ranging from 50 mg/gram to 150 mg/gram (crystal/solvent mixture). The slurry was wet milled with IKA-Works Rotor-Stator high shear homogenizer model T50 with superfine blades at 20-35 m/s, until the mean particle size of SAHA was less than 50 μm and 95% less than 100 μm, while maintaining the temperature at room temperature. The wet-milled slurry was filtered and washed with EtOH/water solvent mixture at room temperature. The wet cake was then dried at 40° C. The final mean particle size of the wet-milled material was less than 50 μm as measured by the Microtrac method as described before.

### Example 6

### Growth of Large Crystals Batch 283

Twenty-four grams of SAHA Polymorph I crystals and 205 ml of 9:1 Ethanol/water solvent mixture were charged into a 500 ml jacketed resin kettle with a glass agitator. The slurry was wet milled to a particle size less than 50 μm at room temperature following the steps of Example 1. The wet-milled slurry was heated to 65° C. to dissolve ˜85% of the solid. The heated slurry was aged at 64-65° C. for 1-3 hours to establish a ˜15% seed bed. The slurry was mixed at an agitator speed range of 100-300 rpm.

The batch was then cooled to 20° C. with one heat-cool cycle: 65° C. to 55° C. in 2 hours, 55° C. for 1 hour, 55° C. to 65° C. over ˜30 minutes, age at 65° C. for 1 hour, 65° C. to 40° C. in 5 hours, 40° C. to 30° C. in 4 hours, 30° C. to 20° C. over 6 hours. The cooled batch was aged at 20° C. for one hour. The batch slurry was filtered and washed with 9:1 EtOH/water solvent mixture at 20° C. The wet cake was dried at 40° C. under vacuum. The dry cake had a final particle size of ˜150 μm with 95% particle size<300 μm per Microtrac method.

Thirty percent of the batch 288 crystals and 70% of the batch 283 crystals were blended to produce capsules containing about 100 mg of suberoylanilide hydroxamic acid; about 44.3 mg of microcrystalline cellulose; about 4.5 mg of croscarmellose sodium; and about 1.2 mg of magnesium stearate.

### Example 7

### Effect of SAHA and Targretin Combinations

### Assay Methods

Initial cytotoxicity and caspase 3/7 assays were run to establish single agent dose response curves in HH cells (CTCL cells; ATCC) treated with Vorinostat (0-30 μM; Merck & Co, Inc.) and Targretin® (0-90 μM) for 24, 48, 72 and 96 hours using ViaLight Plus and Alamar Blue. Data from these assays was used to determine the range of combination concentrations. Both compounds were combined at concentrations close to their IC₅₀ values. Subsequent combination viability/proliferation assays were performed using the ViaLight Plus protocol.

### ViaLight Plus Viability/Proliferation Assay

Costar white with clear bottom 96 well plates (#3603) were seeded with 25,000 cells per well in a volume of 100 μL/well of growth media for each time point. HH cell line growth media included RPMI (GIBCO #) with 10% FBS (GIBCO # SV30014.03), 1% Glutamax (GIBCO # 35050-061), and 1% Penicillin/Streptomycin (GIBCO # 30-002). For this assay, 5× concentrations of Vorinostat (SAHA; Merck & Co, Inc.) and Targretin® were made up for the highest compound concentration and serially diluted passing ⅓ volume compound into ⅔ volume media. For the fixed ratio method, compounds were combined and diluted together serially. For the classical method, a fixed concentration of Targretin® was made in growth media and serial dilutions of Vorinostat were made into it. For each treatment concentration, 25 μL of the appropriate dilution for each compound was added to the corresponding wells. Wells on the outer perimeter of the plate were not used. At each time-point, plates were read using the ViaLight Plus protocol. Luminescence was read using the Victor V plate reader.

### Alamar Blue Viability/Proliferation Assay

Costar black with clear bottom 96 well plates (#3603) were seeded with 25,000 cells per well in a volume of 100 μL/well of growth media for each time point. For this assay, 2×concentrations of Vorinostat and Targretin® were made up for the highest compound concentration and serially diluted passing ⅓ volume compound into ⅔ volume media. For each treatment concentration, 100 μL of the appropriate dilution of each compound was added to the corresponding wells. Wells on the outer perimeter of the plate were not used. Plates were processed according to the Alamar Blue protocol. Briefly, 20 μL of Alamar Blue was added to the 200 μL in each well and allowed to incubate for 6 hours. Fluorescence was read on the Spectra Max plate reader at 530 nm excitation and 590 nm emission.

### Results

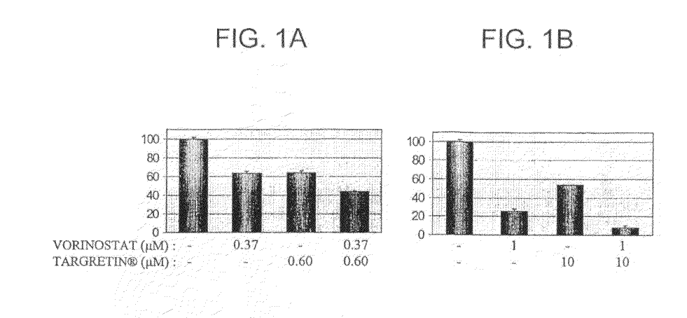

FIGS. 1A-1B summarize the effects of the combination in the concentrations shown. Vorinostat as an agent alone produces an approximate 40% decrease in cell viability (FIG. 1A). Targretin® as a sole agent produces an approximate decrease of 40% in viability (FIG. 1A). When combined, cell viability is decreased by approximately 60%, showing a sub-additive effect (FIG. 1A). A sub-additive effect is also seen with other concentrations (FIG. 1B). There was no antagonistic effect seen in any of the combination concentrations.

### Example 8

### Phase I Clinical Trial of Oral Suberoylanilide Hydroxamic Acid (SAHA) in Combination with Bexarotene in Patients with Advanced Cutaneous T-cell Lymphoma

### Patient Study

A patient study is used to determine the maximum tolerated dose (MTD) of oral SAHA when administered for 28 days in repeated cycles in combination with escalating doses up to 300 mg/m² of Bexarotene in patients with advanced cutaneous T-cell lymphoma. The study is used to assess the safety and tolerability of this regimen and to estimate response rate, time to response, response duration, and time to progression for SAHA and Bexarotene when administered in combination. The study is also used to assess the pharmacokinetics of SAHA and Bexarotene when administered in combination at MTD. The administration of SAHA in combination with Bexarotene at clinically relevant dosages is assessed for sufficient safety and tolerance to permit further study.

### Study Design and Duration

The patient study is an open-label, non-randomized, escalating dose, multicenter, Phase I trial of SAHA in combination with Bexarotene in patients with advanced (stage IB or higher) cutaneous T-cell lymphoma who are refractory to at least one prior systemic treatment and are eligible for Bexarotene therapy. Patients are kept on a 28 day outpatient treatment cycle of oral SAHA and oral Bexarotene until disease progression, intolerable toxicity, or the investigator determines that it is in the best interest of the patient to withdraw. Patients are treated for up to 6 months on this protocol. Patients are seen at regular intervals for assessment of safety (laboratory tests, adverse event assessment, and physical exam) and efficacy. For those who discontinue, a postreatment follow-up visit is conducted within 4 weeks after the last study drug dose or prior to the initiation of new treatment. At baseline, a skin biopsy for correlative studies is obtained. Patients may refuse collection of any correlative sample. Sites also obtain additional skin biopsies at specified intervals for correlative studies.

Patient sample: Approximately 24 to 42 patients are enrolled. A minimum of 3 patients are enrolled at each initial dose level to establish the maximally tolerated dose of the combination therapy. Up to 5 dose levels are planned. Once the MTD for the combination is established, an additional 12 patients are enrolled at the MTD to further gather safety, tolerability and efficacy information, as well as samples for pharmacokinetic analysis of both compounds.

Inclusion criteria: Eligible patients must be ≧18 years with advanced (Stage 1B or higher) progressive, persistent, or recurrent CTCL refractory to at least one systemic treatment. Other eligibility criteria include: histological diagnosis of CTCL documented by biopsy performed within 1 year prior to enrollment; life expectancy >3 months; Eastern Cooperative Oncology Group (ECOG) Performance Status of 0 to 2; ≧4 weeks from prior chemotherapy, biological therapy, radiation therapy, major surgery, or any other investigational therapy; adequate hematologic, hepatic and renal function; and patients must be viable candidates for Bexarotene therapy.

Exclusion criteria: Patients who have had prior treatment with my HDAC inhibitor; Bexarotene treatment within the past 3 months; receiving or within 2 weeks prior to the start of study drug, receives gemfibrozil or other known CYP3A4 inhibitors such as ketoconazole, itraconazole, protease inhibitors, clarithromycin and erythromycin; or known CYP3A4 inducers such as rifampicin, phenyloin, dexamethasone or phenobarbital; an allogeneic transplant; active infection; any systemic steroid treatment that has not been stabilized to the equivalent of ≦10 mg/day prednisone during the 4 weeks immediately prior to the start of study drug; are pregnant or lactating. Patients with a “currently active” second malignancy other than non melanoma skin cancers and carcinoma in situ of the cervix are not eligible. Patients are not considered to have a “currently active” second malignancy if they have completed therapy and are disease free from prior malignancies for 25 years, and are considered to have a less than 30% chance of risk of relapse.

### Dosage/Dosage Form, Route, and Dose Regimen

All doses are administered q.d. orally with food on an outpatient basis in 100-mg increments of SAHA capsules and 75-mg increments of Bexarotene to approximate 150 mg/m² to 300 mg/m² of Bexarotene capsules.

Phase Ia: This is an escalating-dose study with at least 3 patients at each dosing regimen. An additional 3 patients are studied at the MTD attained for the combination. There is no intrapatient dose escalation. Three doses levels of SAHA (200, 300, and 400 mg daily) and three dose levels for Bexarotene (150, 225, and 300 mg/m²) are tested. SAHA is escalated first up to a maximum of 400 mg q.d. maintaining a dose of 150 mg/m² of Bexarotene. The number of Dose Levels tested will depend on when dose limiting toxicity (DLT) is observed.

The dose levels are as follows:

TABLE 3
Dose Levels

| | | Approximate Dose |
|---|---|---|
| Dose | SAHA | of Bexarotene |
| Level | (mg per day) | (mg/m²/day) |
| 1 | 200 | 150 |
| 2 | 300 | 150 |
| 2a* | 200 | 225 |
| 2b* | 200 | 300 |
| 3 | 400 | 150 |
| 3a* | 300 | 225 |
| 3b* | 300 | 300 |
| 4 | 400 | 225 |
| 5 | 400 | 300 |

*If dose level 2 or 3 exceeds MTD, then lower doses of SAHA are tested with escalating doses of Bexarotene as indicated by dose levels 2a/b and 3a/b respectively.

The target dose level for SAHA single-agent therapy in Phase II is 400 mg q.d. for 28 consecutive days, and is the maximum dose of SAHA tested in this trial. As 300 mg/m² is the labeled dose for Bexarotene, it is the maximum dose tested in this trial.

Phase Ib: Twelve patients are administered SAHA q.d. and Bexarotene q.d. at the MTD of the combination. Blood samples for pharmacokinetic measurements are obtained on Day 3 and Day 10 of the first two 28 day cycles.

### Efficacy Measurements

Type of skin lesion (patch, plaque, or tumor) and % involved body surface area (BSA) are assessed using both a Tumor Burden Index (TBI) and a modified Severity Weighted Assessment Tool (mSWAT). For calculation of the TBI, the investigator depicts the area and type of skin lesion on a grid body map. The % of the total body surface area (TBSA) affected by each lesion type is calculated according to the number of grids affected by each lesion type, divided by the total number of grids on the body maps front and back. The modified Severity-Weighted Assessment Tool (mSWAT) uses a transparency of the patient's palm minus the thumb as a reference to equal 1% of TBSA, to directly measure the area of involvement by each lesion type within each of 12 body regions. Both systems assign a weight of 4 for tumor, 2 for plaque and 1 for patch. Severity of pruritus and health-related quality of life are evaluated by the patient at baseline and during each scheduled visit.

### Safety Measurements and Data Analysis

Vital signs, physical examinations, ECOG performance status, electrocardiograms (ECGs), and laboratory safety tests (CBC, comprehensive chemistry panel, APTT, PT/INR urinalysis, liver function, thyroid function, lipid levels) are obtained or assessed prior to drug administration and at designated intervals throughout the study.

Data analysis: This study enrolls ˜24 to 42 patients. Measurements of TBI, mSWAT score, and BSA involvement are tabulated for each patient at every visit. Summary statistics of efficacy (response rate, time to response, response duration, and time to progression) are provided. Pruritus scores are also tabulated for each patient. Patients with complete resolution of pruritus or a ≧3 point drop in pruritus score are summarized. Summary statistics on duration, intensity, and the time to onset of toxicity by dose, are used to assess the adverse effects of the combination therapy. Summary statistics of PK parameter (AUC, Cₘₐₓ, Tₘₐₓ, and t_{1/2}) are provided for SAHA and Bexarotene by sequence and visit day. The difference between the two sequences and the difference between Day 3 and Day 10 within a sequence are explored. Measurements of pharmacodynamic endpoints are summarized. The relationship between safety, pharmacokinetic parameters, and pharmacodynamic endpoints are explored.

### Example 9

### Phase I Clinical Trial of Oral Suberoylanilide Hydroxamic Acid (SAHA) in Combination with Bexarotene in Patients with Advanced Cutaneous T-cell Lymphoma

This study is an open-label, non-randomized, escalating-dose, multicenter, Phase I trial of vorinostat in combination with bexarotene in patients with advanced (Stage 1B or higher) cutaneous T-cell lymphoma who are refractory to at least one prior systemic treatment and are eligible for bexarotene therapy. There are 2 parts to the Phase Ia portion of the study. In Part I, doses of both vorinostat on a mg basis and bexarotene on a mg/m² basis will be escalated. In Part II, the vorinostat dose will be fixed at 400 mg q.d.; doses of bexarotene on a mg basis will be escalated. Patients will be kept on a 28-day outpatient treatment cycle of oral vorinostat and oral bexarotene until disease progression, intolerable toxicity, withdrawal of consent, or the investigator determines that it is in the best interest of the patient to withdraw. Patients will be treated for up to six 28-day cycles on this protocol with the possibility of continuing treatment in the Continuation arm of this study with vorinostat provided by the SPONSOR if there is potential benefit to the patient (i.e., the patient has acceptable toxicity and non-progressive disease, or has any degree of response including complete response (CR)).

Patients will be seen at regular intervals for assessment of safety (laboratory tests, adverse event assessment and physical exam) and efficacy. Response to treatment will be assessed by the investigator by mSWAT score, lymph node measurements as well as other assessments deemed appropriate for the individual patient.

Before the initiation of study drug, a skin biopsy will be requested (Patients may refuse collection of any correlative sample). Additional skin biopsies will be requested at specified intervals for correlative studies.

For patients enrolled in Phase Ia Part I at Dose Level 1, vorinostat will be administered at 200 mg q.d. and bexarotene will be administered at a dose level of 150 mg/m² q.d. If tolerated, dosing for additional cohorts will be escalated as outlined in Section I.E.2.a. For patients enrolled in the Phase Ia Part II, dosing will begin at Dose Level 6 with vorinostat at 400 mg q.d. and bexarotene at 150 mg q.d. The maximum dose of vorinostat for patients enrolled in this study is planned to be 400 mg q.d.; and the maximum dose of bexarotene is planned to be 300 mg/m² for patients enrolled in the Part 1 and 450 mg q.d. bexarotene (not to exceed 300 mg/m² in any individual patient) for patients enrolled in Part II.

Patients will be assessed for safety 1, 2, 4, 6 and 8 weeks after starting the combination treatment of both bexarotene and vorinostat, which encompasses Cycles 1 and 2. Patients with acceptable toxicity may continue to receive additional cycles of treatment.

Following completion of or discontinuation from the study, a post treatment follow-up visit will be conducted within 4 weeks after the last study drug dose or prior to the initiation of new treatment. Patients who withdraw from or complete the study will continue to be followed for safety for 30 days after their last treatment with study medication; thereafter they will be contacted every 2 months for the collection of survival and additional treatment data until the termination of the study, which will occur 6 months after the last patient enrolled has received the first dose of study medication.

### Summary of Study Design for Continuation of Vorinostat

The Continuation arm of the study is an open-label, open-ended, multicenter study to evaluate the safety and tolerability of continued dosing in patients enrolled in the protocol who may benefit from continued therapy with this agent.

Patients will continue to follow the visit schedule for the dose level they have just completed in accordance with the standard of care for their disease and medical condition. The last visit will be treated as the first visit in the continuation arm of the protocol. Separate case report forms will be documented accordingly. Serious adverse experience information will be captured at each visit in addition to nonserious adverse experiences related to drug interruption, discontinuation or dose reduction. Efficacy data will be captured based on Overall Physician's Assessment. Efficacy and safety (including laboratory) evaluations will be performed per clinical standard of care for the given disease state to justify the patient's continuation on the study per clinical presentation. These assessments may be performed at 4-week intervals but no more than every 6 weeks, and will be documented on case report forms. Patients must be taken off study drug for disease progression or development of unacceptable toxicity.

### Investigational Study Drugs

At each dose level, the appropriate numbers of 100-mg capsules of vorinostat and 75-mg capsules of bexarotene are to be administered q.d. orally in repeated 28-day cycles.

During the dosing period, the capsules should be taken with food (within 30 minutes following a meal), whenever possible. The total dose consumed at any one time should not exceed the assigned dose; missed doses should not be made up.

Sufficient drug for treatment until the next scheduled study visit will be dispensed at each visit. Any unused drug should be returned to the site at the completion of the dosing period of the cycle. A capsule count will be performed at each study visit to monitor compliance.

### Dose Schedules for Patients Enrolled in Part I

In Part I (original protocol), up to three dose levels of vorinostat (200, 300, and 400 mg daily) and up to three dose levels of bexarotene (150, 225, and 300 mg/m²) will be tested (Table 3). If tolerated, vorinostat will be escalated first, maintaining a dose of 150 mg/m² of bexarotene. The number of Dose Levels tested will depend on the dose level at which DLTs are observed.

The starting dose level of vorinostat (Dose Level 1) will be 200 mg q.d and the starting dose of bexarotene will be 150 mg/m² q.d. for 28 day cycles.

TABLE 4
Dose Levels

| | | Approximate Dose |
|---|---|---|
| Dose | Vorinostat | of Bexarotene |
| Level | (mg per day) | (mg/m²/day) |
| 1 | 200 | 150 |
| 2 | 300 | 150 |
| 2a* | 200 | 225 |
| 2b* | 200 | 300 |
| 3 | 400 | 150 |
| 3a* | 300 | 225 |
| 3b* | 300 | 300 |
| 4 | 400 | 225 |
| 5 | 400 | 300 |

*If dose level 2 or 3 exceeds the MTD, then lower doses of vorinostat will be tested with escalating doses of bexarotene as indicated by dose levels 2a/b and 3a/b, respectively. If 150 mg/m²/day bexarotene is not tolerated at Dose Level 2 or 3, the investigator may administer 100 mg/m²/day of bexarotene for that patient.

### Dose Schedules for Patients Enrolled in Part II

In Part II, 400 mg vorinostat q.d. will be administered at all dose levels. Up to five dose levels of bexarotene (150, 225, 300, 375 and 450 mg q.d.) will be tested. The number of Dose Levels tested will depend on the dose level at which DLTs are observed.

Recently described strategies for supportive care to minimize the potential lipid and thyroid function changes associated with bexarotene use, as described in Section I.E.2.a3.a of the amended protocol, will be implemented.

At the initial dose level of Part II (Dose Level 6), the vorinostat dose will be 400 mg q.d and the dose of bexarotene will be 150 mg q.d. for six 28-day cycles of combination therapy. For subsequent dose levels, bexarotene will initially be given at 150 mg q.d., and titrated in patients on a 28-day basis up to the target dose for that dose level (Table 4) in order to lessen the likelihood of bexarotene-related toxicities.

TABLE 5
Dose Levels

| | Vorinostat | Bexarotene | Bexarotene | Bexarotene |
|---|---|---|---|---|
| Dose | (mg/day) | (mg/day) | (mg/day) | (mg/day) |
| Level | Cycles 1-6 | Cycle 1 | Cycle 2 | Cycle 3-6 |
| 6 | 400 | 150 | 150 | 150 |
| 7 | 400 | 150 | 225 | 225 |
| 8 | 400 | 150 | 225 | 300 |
| 9 | 400 | 150 | 300 | 375 |
| 10 | 400 | 150 | 300 | 450 |

If 150 mg q.d. bexarotene is not tolerated, the investigator may administer 75 mg q.d. of bexarotene. Alternatively, vorinostat alone may be administered.

Once the MTD of Part II is determined for vorinostat and bexarotene in combination, 12 additional patients will be enrolled at the MTD of the combination in the Phase Ib portion of the study, and PK sampling will be conducted (see Pharmacokinetic Measurements under I.F.2).

### Part II: Incorporation of Supportive Care Guidelines for Bexarotene Treatment

For patients enrolled in Part II, the supportive care guidelines described below (Assaf et al., 2006) should be instituted to minimize potential lipid and thyroid effects of bexarotene:

Patients will be treated with a lipid-lowering regimen, preferably fenofibrate (suggested dose of 145-200 mg daily), for at least one week prior to administration of the first dose of bexarotene. Fenofibrate dose should be reduced to 100 mg (or 50 mg, if necessary) daily if creatinine is >1.5 mg/dL (0.133 μmol/L) or patient has nephrotic syndrome.

For patients with coronary heart disease who are likely to have active atherosclerotic plaques, or higher than normal LDL cholesterol levels, low-dose statin therapy may be started at least 3 days before the first dose of bexarotene. For optimal effect, fibrate should be administered in the morning and statins should be administered in the evening.

Vorinostat should be given for at least 1 week prior to bexarotene therapy and started at the same time as or after lipid-lowering therapy has been initiated. After one week of vorinostat in combination with a lipid-lowering regimen, bexarotene may be administered. A lipid profile should be obtained at the time of initiation of bexarotene, and the lipid profile measurements (triglycerides, HDL, LDL cholesterol) obtained at this time must be normal in order for the patient to continue receiving bexarotene in this portion of the study.

Concomitant with the first dose of bexarotene, low dose thyroxine (e.g. 0.05 mg levothyroxine q.d.) therapy should be started prophylactically.

Thyroxine and lipid lowering therapy doses should be adjusted as needed throughout treatment.

Bexarotene may be titrated to the targeted dose for each subsequent cycle (if greater than 150 mg) if lipid levels and thyroid function tests (free T4 levels) remain normal.

For the Phase Ib portion of the study, both the lipid-lowering regimen and levothyroxine therapy (0.05 mg/day) should be initiated at least one week prior to initiation of bexarotene or vorinostat therapy. The administration of levothyroxine for this one-week period is to allow levothyroxine to reach approximate steady state before PK samples are obtained.

### Definition of Dose-Limiting Toxicity

Toxicity will be graded as per CTCAE guidelines. A dose-limiting toxicity (DLT) is defined as any of the following:
A drug-related CTCAE Grade 3 or 4 non-hematologic event not manageable by supportive care or non-prohibited therapies, except the following:
   alopecia
   if the baseline ALT or AST level was grade 2 and the increase in AST/ALT level is ≦2.5×ULN
   inadequately treated diarrhea, nausea, or vomiting
Grade 3-4 neutropenia with fever ≧38.5° C. and/or with an infection requiring antibiotic or antifungal treatment
Grade 4 neutropenia lasting at least 5 days,
Grade 4 thrombocytopenia OR platelet count<25,000μ/L

Dose escalation will be determined based on the occurrence of DLTs. For the purposes of determining whether to advance the Dose Level, DLTs will be counted by patient (i.e., a patient who experiences more than 1 DLT will be counted only once). For Part I, DLTs observed during the first treatment cycle will be counted. For Part II, DLTs will be counted during the initial cycle of combination treatment up through completion of the first cycle of the highest combination dose for that Dose Level.

### Determination of the Maximum Tolerated Dose

### Part I

The timing for enrollment and dose escalation rules for Part I are as follows:

Part I will proceed stepwise into each Dose Level after patients have completed a 28 day cycle of combination therapy with either no DLTs observed (in 3 patients) or only I DLT observed (in 6 patients).

At each dose level, three patients will initially be enrolled, treated, and observed for 1 full cycle (28 days of combination treatment).
If no DLTs are observed in the first cycle, then 3 new patients will be enrolled at the next higher dose level (up to Dose Level 5).
If 1 of the first 3 patients experiences a DLT, then an additional 3 patients will be enrolled, treated, and observed at that dose level for 1 full cycle (28 days).
   If no additional patients experience a DLT (i.e., only 1 of 6 patients experiences a DLT), then 3 new patients will be enrolled at the next higher dose level (up to Dose Level 5).
If 1 or more additional patients experiences a DLT (i.e., total of ≧2 of 6 patients), then the MTD has been exceeded and additional patients will be enrolled at the previous dose level as needed so that a total of 6 patients will have been enrolled at the MTD.
If 2 or more of the first 3 patients at a given dose level experience a DLT, then the MTD has been exceeded and additional patients will be enrolled at the previous dose level as needed so that a total of 6 patients will have been enrolled at the MTD.

### Part II

For any given dose combination in Part II, additional patients will be enrolled at a given dose level if ≧16% and ≦33% of total patients that have received that dose have had a DLT. If ≧33% of patients that have received that specific dose have had a DLT, then the MTD has been exceeded.

Dose Level 6: Three patients may enroll immediately. If 1 DLT is seen, then an additional 3 patients will enroll in this cohort. If 2 or more DLTs are seen, then the MTD has been exceeded.
Dose Level 7: Three patients may enroll after 3 patients at Dose Level 6 have completed one 28 day cycle of combination therapy with no DLTs or 6 patients at Dose Level 6 have completed one 28 day cycle of combination therapy with 1 DLT. Additional patients will be enrolled at Dose Level 7 if 1 DLT is seen in Cycle 2. If 2 or more DLTs are seen in Cycle 2, then the MTD has been exceeded.
Dose Level 8: Three patients may enroll after 3 patients at Dose Level 7 have completed one 28 day cycle of combination therapy with less than 33% of the total number of patients that have received the 400 mg vorinostat/150 mg bexarotene combination having had a DLT. Additional patients will be enrolled at Dose Level 8 if 1 DLT is seen in Cycle 3. If 2 or more DLTs are seen in Cycle 3 at Dose Level 8, then the MTD has been exceeded.
Dose Level 9: Three patients may enroll after the second 28 day cycle of combination treatment at Dose Level 8 has been completed with ≦1 DLT observed in Cycle 3. Additional patients will be enrolled at Dose Level 9 if 1 DLT is seen in Cycle 2 or 3. If 2 or more DLTs are seen in Cycle 2 or 3, then the MTD has been exceeded.
Dose Level 10: Three patients may enroll after 3 patients at Dose Level 9 have completed one 28 day cycle of combination therapy with less than 33% of the total number of patients that have received the 400 mg vorinostat/150 mg bexarotene combination having had a DLT. Additional patients will be enrolled at Dose Level 10 if 1 DLT is seen in Cycle 3. If 2 or more DLTs are seen in Cycle 3 in Dose Level 10, then the MTD has been exceeded.

Once the MTD of Part II is determined for vorinostat and bexarotene in combination, 12 additional patients will be enrolled at the MTD of the combination in the Phase Ib portion of the study, and PK sampling will be conducted (see Pharmacokinetic Measurements under I.F.2).

### Dose Modification and Treatment Delay

The NCI Common Terminology for Adverse Events (CTCAE, Version 3.0) guide will be used to assess adverse events. Vorinostat and/or bexarotene may be held in the presence of Grade 3 or 4 non-drug related toxicity if the physician feels it is unsafe to continue the administration of vorinostat and/or bexarotene.

In the presence of Grade 3 to 4 drug-related non-hematologic toxicity, vorinostat and/or bexarotene should be held until the toxicity resolves to Grade 1 or less. Interruption of study drug(s) should be assessed on a case by case basis based on the study drug's probable causality of the adverse event.

In the presence of Grade 3 or 4 lipid-related adverse event or thyroid function adverse event, bexarotene should be held and vorinostat may be held at the discretion of the investigator.

In the instance of Grade 3 anemia or thrombocytopenia, vorinostat and bexarotene may be continued if, in the opinion of the investigator, the toxicity can be managed.

After recovery from drug-related toxicity that resulted in a dose delay, dose modification will proceed by resumption of dosing at a dose equal to or lower than that previously administered to that patient, unless in the opinion of the investigator and the SPONSOR, dose modification is not necessary. Patients who have recovered from a toxicity that resulted in a dose modification may be allowed to return to their originally assigned dose following discussion between the investigator and the SPONSOR.

If toxicity occurs at Dose Level 6 that may be related to vorinostat, the dose of vorinostat may be reduced to 300 mg q.d. If a second dose reduction of vorinostat is needed, the dose may be reduced to 300 mg q.d. 5 days on/2 days off. If bexarotene-related toxicity occurs at Dose Level 1, patients will be allowed to discontinue bexarotene and then in subsequent cycles receive intrapatient dose escalation of vorinostat to 300 mg followed by 400 mg, if tolerated. If bexarotene-related toxicity occurs at Dose Level 6, the patient may receive 400 mg q.d. vorinostat only or 400 mg q.d. vorinostat and 75 mg q.d. bexarotene.

While this invention has been particularly shown and described with references to the embodiments thereof, it will be understood by those skilled in the art that various changes in form and details may be made therein without departing from the meaning of the invention described. The scope of the invention encompasses the claims that follow.

What is claimed is:

1. A method of treating cancer in a subject in need thereof comprising administering to the subject a histone deacetylase inhibitor, suberoylanilide hydroxamic acid (SAHA), represented by the structure: 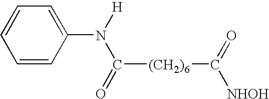
or a pharmaceutically acceptable salt or hydrate thereof, and a retinoid agent, 4-[1-(5,6,7,8-Tetrahydro-3,5,5,8,8-pentamethyl-2-naphthalenyl)ethenyl]benzoic acid (3-methyl TTNEB) (Targretin), represented by the structure: 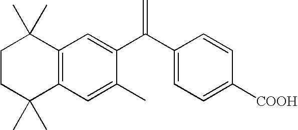
or a pharmaceutically acceptable salt or hydrate thereof, wherein the histone deacetylase inhibitor and the retinoid agent are administered in amounts effective for treating the cancer.

2. The method of claim 1, wherein the histone deacetylase inhibitor is administered prior to administering the retinoid agent.

3. The method of claim 1, wherein the histone deacetylase inhibitor and the retinoid agent are administered orally.

4. The method of claim 3, wherein the cancer is selected from the group consisting of a leukemia, a lymphoma, a myeloma, a sarcoma, a carcinoma, a solid tumor or any combination thereof.

5. The method of claim 3, wherein the cancer is a cutaneous T-cell lymphoma.

6. The method of claim 5, wherein SAHA is pre-administered I week prior to the concurrent administration of SAHA and Targretin.

7. The method of claim 6, wherein SAHA is administered 400 mg once a day in the pre-administration and concurrent administration.

8. The method of claim 7, wherein in the concurrent administration, Targretin is administered at 150 mg per day.

9. The method of claim 7, where the concurrent administration is for six 28-day cycles.

10. The method of claim 6, wherein in the concurrent administration of SAHA and Targretin, SAHA is administered 400 mg once a day for six 28-day cycles, Targretin is administered at 150 mg per day for the first 28-day cycle, and at 225 mg per day for the second to sixth 28-day cycle.

11. The method of claim 6, wherein in the concurrent administration of SAHA and Targretin, SAHA is administered 400 mg once a day for six 28-day cycles, Targretin is administered at 150 mg per day for the first 28-day cycle, at 225 mg per day for the second 28-day cycle, and at 300 mg per day for the third to sixth 28-day cycle.

12. The method of claim 6, wherein in the concurrent administration of SAHA and Targretin, SAHA is administered 400 mg once a day for six 28 day cycles, Targretin is administered at 150 mg per day for the first 28-day cycle, at 300 mg per day for the second 28-day cycle, and at 375 mg per day for the third to sixth 28-day cycle.

13. The method of claim 6, wherein in the concurrent administration of SAHA and Targretin, SAHA is administered 400 mg once a day for six 28-day cycles, Targretin is administered at 150 mg per day for the first 28-day cycle, at 300 mg per day for the second 28-day cycle, and at 450 mg per day for the third to sixth 28 day cycle.

14. The method of claim 13, wherein a lipid-lowering agent is administered during or before the pre-administration period, or a combination thereof.

15. The method of claim 14, wherein the lipid-lowering agent is fenofibrate.

16. The method of claim 13, wherein thyroxine is administered at the start of the concurrent administration period.

17. The method of claim 16, wherein the thyroxine is levothyroxine.

18. A pharmaceutical composition comprising a histone deacetylase inhibitor, suberoylanilide hydroxamic acid (SAHA), represented by the structure: 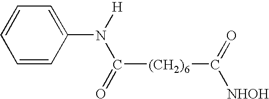
or a pharmaceutically acceptable salt or hydrate thereof, and a retinoid agent, 4-[1-(5,6,7,8-Tetrahydro-3,5,5,8,8-pentamethyl-2-naphthalenyl)ethenyl]benzoic acid (3-methyl TTNEB) (Targretin), represented by the structure: 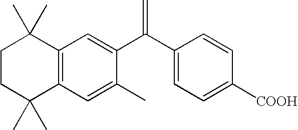
or a pharmaceutically acceptable salt or hydrate thereof.

19. The pharmaceutical composition of claim 18, wherein the composition is formulated for oral administration.

20. The pharmaceutical composition of claim 19 that comprises 100 mg of SAHA and 75 mg of Targretin.
